(19) United States
(12) Patent Application Publication
(10) Pub. No.: US 2016/0242956 A1
(43) Pub. Date: Aug. 25, 2016

(54) PRE AND POST ANESTHETIC COOLING DEVICE AND METHOD

(71) Applicant: JENNIFER MARIE PILBY GOMEZ, ALPINE, CA (US)

(72) Inventor: JENNIFER MARIE PILBY GOMEZ, ALPINE, CA (US)

(21) Appl. No.: 15/051,665

(22) Filed: Feb. 24, 2016

Related U.S. Application Data
(60) Provisional application No. 62/120,629, filed on Feb. 25, 2015.

(51) Int. Cl. A61F 7/00; A61F 7/12

(52) U.S. Cl. CPC A61F 7/007; A61F 7/12; A61F 2007/0017; A61F 2007/0075; A61F 2007/0087

(57) ABSTRACT

Thermoelectric anesthetic cooling devices allow for temperature controlled cooling of the skin/epidermis or mucous membrane/mucosa, to alleviate pain associated with medical treatment such as injections and skin ablation applied to the human body. The cooling device comprises a body that efficiently conducts heat away from the thermoelectric section of the device. The device body comprises a proximal gripping end (i.e., handle) connected to a distal head section by a neck section.

### CROSS-REFERENCE TO RELATED APPLICATIONS

This application claims the benefit of U.S. Provisional Application No. 62/120,629, filed on Feb. 25, 2015.

### FIELD OF THE INVENTION

The present invention pertains to a device and a method for temperature controlled cooling of the skin/epidermis or mucous membrane/mucosa. In particular, the present invention is directed to devices and techniques to alleviate pain associated with medical treatments, such as injections and skin ablation applied to the human body.

### BACKGROUND OF THE INVENTION

Nerve conduction block is an important technique for use in the medical and dental fields. Currently, nerve conduction block is achieved by application of chemical compounds/formulations such as topical and local anesthesia, or via thermal application such as ice.

Topical anesthetics reversibly block nerve conduction near their site of administration, thereby producing temporary loss of sensation in a limited area. Nerve impulse conduction is blocked by decreasing nerve cell membrane permeability to sodium ions, possibly by competing with calcium-binding sites that control sodium permeability. This change in permeability results in decreased depolarization and an increased excitability threshold that, ultimately, prevents the nerve action potential from forming.

Disadvantages to topical anesthetic are variability in systemic absorption, toxicity, poor absorption through intact skin, allergic reactions and adverse effects. Adverse effects are usually caused by high plasma concentrations of topical anesthetics that typically result from excessive exposure caused by application to abraded or torn skin. Possible adverse effects include the following: burning or stinging that may occur local to the administration site; and oral viscous lidocaine that may cause systemic toxicity, particularly with repeated use in infants or children. In the Central Nervous System (CNS), high plasma concentration initially produces CNS stimulation (including seizures), followed by CNS depression (including respiratory arrest). The CNS stimulatory effect may be absent in some patients, particularly when amides (e.g., tetracaine) are administered. Solutions that contain epinephrine may add to the CNS stimulatory effect. In Cardiovascular applications, high plasma levels typically depress the heart and may result in bradycardia, arrhythmias, hypotension, cardiovascular collapse, and cardiac arrest. Local anesthetics that contain epinephrine may cause hypertension, tachycardia, and angina, while gag-reflex suppression may occur with oral administration.

Other body systems can also experience adverse effects such as transient burning sensation, skin discoloration, swelling, neuritis, tissue necrosis and sloughing, and Methemoglobinemia (with prilocaine).

The United States Food and Drug Administration (FDA) has issued an advisory regarding risk of serious adverse effects with the use of topical anesthetics for cosmetic procedures. Life-threatening adverse effects have occurred following topical anesthetic application over large surface areas of the body. Two women experienced seizures, coma, and death following applying topical anesthetics to their legs with an occlusive dressing before laser hair removal.

Studies indicate that lowering the body temperature at an injured site can reduce swelling and pain while promoting healing. A common technique to provide relief to an injured site or analgesia before injection is to apply ice, usually in an ice pack. Although ice has the advantage of being inexpensive and readily available, it is not healthy to apply ice to the skin for prolonged periods of time. Another disadvantage of using ice is that it can cause cellular damage if it is applied for more than a few minutes in one area due to temperatures below 0° C. As a result, ice only cools the upper surface of the skin and deep penetration of the cooling process does not take place.

Achieving pain free injections can be difficult for dentists, especially through taut tissue such as in the hard palate of the mouth.

Fear-related behaviors have long been recognized as the most difficult aspect of patient management and can be a barrier to good care. Anxiety is one of the major issues in the dental treatment of children, and the injection is the most anxiety-provoking procedure for both children and adults.

Fears of dental injections remain a clinical problem often requiring cognitive behavioral psychology counseling and sedation in order to carry out needed dental treatment. High levels of dental anxiety and fear have been reported in many industrialized Western societies. There is considerable evidence that dental fear is related to poorer oral health, reduced dental attendance and increased treatment stress for the attending dentist. Indeed, fear of needles and the treatment of injection fear has been an important focus. Needle fear, in particular, is a major issue given that the delivery of local anesthesia via injection is the central plank of pain relief techniques in dentistry and dentists as well as patients often avoiding difficult injections as a consequence, resulting in poor pain control.

Thus, there is a need for methods to avoid the invasive, and often painful, nature of the injection, and in particular, to find more comfortable and pleasant means for anesthesia before dental procedures.

Application of a cooling device to control pain associated with procedures such as laceration repair may avoid the need for infiltrative local anesthesia injections and associated pain from the injections. Cooling also avoids the risk of wound margin distortion that exists with infiltrative injection administration.

U.S. Pat. No. 7,981,080 discloses a cooling device that is a bulky hypodermic injection arrangement with an opening that does not allow for direct contact to the skin with the cooling plate. However, the bulkiness does not allow for visualization of the injection site, thereby making it difficult for the clinician to use.

U.S. Pat. No. 8,758,419 (the '419 patent) discloses a contact cooler that uses recirculating temperature controlled fluid. The '419 patent discloses a handle attached by a hose to a large control unit where the thermoelectric plates and fluid are housed, however, this unit is very bulky due to the control unit size. In addition, the head of the unit is too large to be used intra-orally and has a reduced cooling capacity due to the fluid having to travel a distance from the cooling plates in the unit to the source it is cooling.

Thus, there is a need for a device and method to provide pain relief that does not have the aforementioned shortcomings.

### SUMMARY OF THE INVENTION

The present invention is directed to a novel thermoelectric device and method for transient nerve cooling block of the peripheral nerves system. The thermoelectric cooling device of the present invention allows for temperature controlled cooling of the skin/epidermis or mucous membrane/mucosa, in order to alleviate pain associated with medical treatment such as injections and skin ablation applied to the human body. The cooling device comprises a body that efficiently conducts heat away from the thermoelectric section of the device. The cooling device body comprises a proximal gripping end (i.e., handle) connected to a distal head section by a neck section.

In one embodiment, the user (i.e., a clinician) first numbs the target tissue by placing the cooling surface of the thermoelectric assembly located in the distal head section of the thermoelectric cooling device against the tissue. The cool side covered with a hygienic barrier sleeve contacts the tissue for 35 seconds or less until the tissue reaches 6° C. or less. Then, the clinician removes the intra-oral or extra-oral thermoelectric device and inserts the hypodermic needle into the target tissue, thereby allowing for a pain-free experience for the patient.

In another embodiment, the user (i.e., a clinician) first numbs the target tissue by placing the cooling surface of the thermoelectric assembly located in the handle section of the thermoelectric cooling device against the tissue. The cool side covered with a hygienic barrier sleeve contacts the tissue for 35 seconds or less until the tissue reaches 6° C. or less. Then, the clinician removes the intra-oral or extra-oral thermoelectric device and inserts the hypodermic needle into the target tissue allowing for a pain-free experience for the patient.

In an embodiment, the present invention is directed to a thermoelectric anesthetic cooling device comprising a device body comprising a handle; a distal head end, wherein the distal head end comprising a head section and a neck section; a proximal end; and a plurality of thermally conductive layers for dissipating heat from the cooling device. The cooling device of the present invention further comprises a thermoelectric assembly, wherein the thermoelectric assembly comprises a plurality of thermoelectric plates; and the thermoelectric assembly is configured to cool a patient skin target tissue; and an electronic assembly that is configured to energize the thermoelectric assembly, wherein the electronic assembly is electrically interconnected to the thermoelectric assembly; and wherein the head section is configured to support or contain the thermoelectric assembly.

In another embodiment, the thermoelectric anesthetic cooling device of is an intra-oral device. In yet another embodiment, the cooling device is an extra-oral device.

In yet another embodiment, the thermoelectric anesthetic cooling device comprises a continuous body; and wherein the handle, the head section, and the distal head end together comprise a single piece of material.

In still another embodiment, the thermoelectric assembly comprises a plurality of thermoelectric plates that comprises at least one inner thermoelectric plate; at least one outer thermoelectric plate; and a layer of semiconductor material arranged with spaced apart portions of P-type semiconductor and N-type semiconductor material, wherein the layer is disposed between the at least one inner thermoelectric plate and the at least one outer thermoelectric plate.

In one embodiment, at least one outer thermoelectric plate provides cooling, and wherein a user contacts a patient's target skin tissue with the at least one outer thermoelectric plate.

In another embodiment, the plurality of thermoelectric plates comprises ceramic, glass, porcelain, or composite polymer; and In one embodiment, the plurality of thermoelectric plates comprises ceramic, glass, porcelain, or composite polymer; and the thermoelectric plates are cascaded, are in a multi-stage thermoelectric cooler, or are stacked on top of each other. In yet another embodiment, the heat generated during operation of the plurality of thermoelectric plates is dissipated by a first thermally conductive layer located within interior of the body; and a second thermally conductive layer located on the exterior of the distal head end and the exterior of the handle.

In another embodiment, thermoelectric plates are cascaded, are in a multi-stage thermoelectric cooler, or are stacked on top of each other. In yet another embodiment, the heat generated during operation of the plurality of thermoelectric plates is dissipated by a first thermally conductive layer located within interior of the body; and a second thermally conductive layer located on the exterior of the distal head end and the exterior of the handle.

In still another embodiment, the first thermally conductive layer comprises copper, aluminum alloys, water-copper heat pipe, or copper heat pipe; and wherein the second thermally conductive layer comprises tellurium copper rod, aluminum, aluminum alloys, copper, beryllium oxide, diamond, aluminum nitride, or one or more combinations thereof.

In one embodiment, the plurality of thermally conductive layers comprises tellurium copper rod, aluminum, magnesium and aluminum alloys, polymers, ceramics, fibers, copper, beryllium oxide, diamond, aluminum nitride, or one or more combinations thereof.

In yet another embodiment, the device further comprises a heat sink selected from a water-copper heat pipe, or a fan for removing accumulated heat in the thermoelectric assembly.

In still one embodiment, the present invention is directed to a method of selective localized cooling of a patient target tissue using a thermoelectric anesthetic cooling, wherein the method comprising energizing the thermoelectric anesthetic cooling device; activating cooling function of the thermoelectric anesthetic cooling device by energizing the plurality of the thermoelectric plates by the electronic assembly; placing a cool surface of the thermoelectric plates against the patient target tissue; cooling the patient target tissue for a desired time and temperature; and removing the device from the patient target tissue.

In yet another embodiment, the present invention is directed to a thermoelectric anesthetic cooling device comprising a device body comprising a handle; a distal head end, wherein the distal head end comprises a head section and a neck section; a proximal end; and a plurality of thermally conductive layers for dissipating heat from the cooling device; a thermoelectric assembly, wherein the thermoelectric assembly comprises a plurality of thermoelectric plates; and the thermoelectric assembly is configured to cool a patient skin target tissue; and an electronic assembly configured to energize the thermoelectric assembly, wherein the electronic assembly is electrically interconnected to the thermoelectric assembly; and wherein the handle is configured to support or contain the thermoelectric assembly. In one embodiment, the thermoelectric anesthetic cooling device is an intra-oral device and in another embodiment, the thermoelectric anesthetic cooling device is an extra-oral device.

In one embodiment, the thermoelectric anesthetic cooling device comprises a continuous body, wherein the handle, the head section and the distal head end comprise a single piece of material.

In another embodiment, the plurality of thermoelectric plates comprises at least one inner thermoelectric plate; at least one outer thermoelectric plate; and a layer of semiconductor material arranged with spaced apart portions of P-type semiconductor and N-type semiconductor material, wherein the layer is disposed between the at least one inner thermoelectric plate and the at least one outer thermoelectric plate.

In one embodiment, the at least one inner thermoelectric plate provides cooling; the at least one inner thermoelectric plate is adjacent to a first thermally conductive layer; and wherein a user contacts a patient's target skin tissue through a cool surface at the head section of the device.

In another embodiment, the plurality of thermoelectric plates comprises ceramic, glass, porcelain or composite polymer, and the thermoelectric plates are cascaded, are in a multi-stage thermoelectric cooler, or are stacked on top of each other.

In one embodiment, the heat generated during operation of the plurality of thermoelectric plates is dissipated by a second thermally conductive layer located on the exterior of the distal head end and the exterior of the handle.

In one embodiment, the first thermally conductive layer comprises copper, aluminum alloys, water-copper heat pipe, or copper heat pipe.

In yet another embodiment, the second thermally conductive layer comprises tellurium copper rod, aluminum, aluminum alloys, copper, beryllium oxide, diamond, aluminum nitride, or one or more combinations thereof.

In one embodiment, the plurality of thermally conductive layers comprises tellurium copper rod, aluminum, magnesium and aluminum alloys, polymers, ceramics, fibers, copper, beryllium oxide, diamond, aluminum nitride, or one or more combinations thereof. In still another embodiment, the cooling device further comprises a heat sink selected from a water-copper heat pipe, and a fan for removing accumulated heat in the thermoelectric assembly.

In one embodiment, the present invention is directed to an intra-oral thermoelectric anesthetic cooling device comprising a device body that comprises a handle; a distal head end, wherein the distal head end comprises a head section, and a neck section; a proximal end; and a plurality of thermally conductive layers for dissipating heat from the thermoelectric anesthetic cooling device; and wherein the body comprises a continuous body, and wherein the handle, the head section and the distal head end comprise a single piece of material. In one embodiment, the cooling device further comprises a thermoelectric assembly, wherein the thermoelectric assembly comprises a plurality of thermoelectric plates, wherein the plurality of thermoelectric plates comprises at least one inner thermoelectric plate; at least one outer thermoelectric plate; and a layer of semiconductor material arranged with spaced apart portions of P-type semiconductor and N-type semiconductor material, wherein the layer is disposed between the at least one inner thermoelectric plate and the at least one outer thermoelectric plate.

In one embodiment, the at least one outer thermoelectric plate provides cooling, and wherein a user contacts a patient's skin target tissue through the at least one outer thermoelectric plate; and the thermoelectric assembly is to configured to cool a patient's target skin surface. The cooling device further comprises an electronic assembly configured to energize the thermoelectric assembly, wherein the electronic assembly is electrically interconnected to the thermoelectric assembly; and wherein the head section is configured to support or contain the thermoelectric assembly.

In another embodiment, the plurality of thermoelectric plates comprises ceramic, glass, porcelain or composite polymer; and the thermoelectric plates are cascaded, are in a multi-stage thermoelectric cooler, or are stacked on top of each other.

In still another embodiment, the heat generated during operation of the plurality of thermoelectric plates is dissipated by a first thermally conductive layer located within interior of the body; and a second thermally conductive layer located on the exterior of the distal head end and the exterior of the handle.

In one embodiment, the first thermally conductive layer comprises copper, aluminum alloys, water-copper heat pipe, or copper heat pipe; and wherein the second thermally conductive layer comprises tellurium copper rod, aluminum, aluminum alloys, copper, beryllium oxide, diamond, aluminum nitride, or one or more combinations thereof.

In another embodiment, the plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

In one embodiment, the cooling device further comprises a heat sink selected from a water-copper heat pipe, and a fan for removing accumulated heat in the thermoelectric assembly.

In still another embodiment, the present invention is directed to an intra-oral thermoelectric anesthetic cooling device comprising a device body that comprises a handle; a distal head end, wherein the distal head end comprises a head section and a neck section; a proximal end; and a plurality of thermally conductive layers for dissipating heat from the cooling device; and wherein the body comprises a continuous body, and wherein the handle, the head section and the distal head end comprise a single piece of material; and a thermoelectric assembly, wherein the thermoelectric assembly comprises a plurality of thermoelectric plates.

In an embodiment, the plurality of thermoelectric plates comprises at least one inner thermoelectric plate; at least one outer thermoelectric plate; and a layer of semiconductor material arranged with spaced apart portions of P-type semiconductor and N-type semiconductor material, wherein the layer is disposed between the inner thermoelectric plates and the outer thermoelectric plates. In an embodiment, the at least one inner thermoelectric plate provides cooling, the at least one inner thermoelectric plate is adjacent to a first thermally conductive layer; and wherein a user contacts the patient's target skin tissue through a cool surface at the head section of the device.

In yet another embodiment, the thermoelectric assembly is configured to cool a patient's target skin surface. In an embodiment, the cooling device further comprises an electronic assembly configured to energize the thermoelectric assembly, wherein the electronic assembly is electrically interconnected to the thermoelectric assembly; and wherein the handle is configured to support or contain the thermoelectric assembly.

In one embodiment, the plurality of thermoelectric plates comprises ceramic, glass, porcelain or composite polymer, and the thermoelectric plates are cascaded, are in a multi-stage thermoelectric coolers, or are stacked on top of each other.

In another embodiment, the heat generated during operation of the plurality of thermoelectric plates is dissipated by a second thermally conductive layer located on the exterior of the distal head end and the exterior of the handle. In one embodiment, the first thermally conductive layer comprises copper, aluminum alloys, water-copper heat pipe, or copper heat pipe. In another embodiment, the second thermally conductive layer comprises tellurium copper rod, aluminum, aluminum alloys, copper, beryllium oxide, diamond, aluminum nitride, or one or more combinations thereof.

In one embodiment, the plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

In yet another embodiment, the cooling device further comprises a heat sink selected from a water-copper heat pipe, and a fan for removing accumulated heat in the thermoelectric assembly.

In yet another embodiment, the present invention is directed to an extra-oral thermoelectric anesthetic cooling device comprising a device body comprising a handle; a distal head end, wherein the distal head end comprises a head section, and a neck section; a proximal end; and a plurality of thermally conductive layers for dissipating heat from the cooling device; and wherein the body comprises a continuous body, and wherein the handle, the head section and the distal head end comprise a single piece of material. In one embodiment, the cooling device further comprises a thermoelectric assembly, wherein the thermoelectric assembly comprises a plurality of thermoelectric plates, wherein the plurality of thermoelectric plates comprises at least one inner thermoelectric plate; at least one outer thermoelectric plate; and a layer of semiconductor material arranged with spaced apart portions of P-type semiconductor and N-type semiconductor material, wherein the layer is disposed between the at least one inner thermoelectric plate and the at least one outer thermoelectric plate.

In an embodiment, the at least one outer thermoelectric plate provides cooling, and wherein a user contacts the patient's target skin tissue through the at least outer thermoelectric plate. In one embodiment, thermoelectric assembly is configured to cool a patient's target skin surface. In one embodiment, the cooling device further comprises an electronic assembly configured to energize the thermoelectric assembly, wherein the electronic assembly is electrically interconnected to the thermoelectric assembly; and the head section is configured to support or contain the thermoelectric assembly.

In one embodiment, the plurality of thermoelectric plates comprises ceramic, glass, porcelain or composite polymer; and the thermoelectric plates are cascaded, are in a multi-stage thermoelectric cooler, or are stacked on top of each other. In one embodiment, the heat generated during operation of the plurality of thermoelectric plates is dissipated by a first thermally conductive layer located within interior of the body; and a second thermally conductive layer located on the exterior of the distal head end and the exterior of the handle.

In one embodiment, the first thermally conductive layer comprises copper, aluminum alloys, water-copper heat pipe, or copper heat pipe; and wherein the second thermally conductive layer comprises tellurium copper rod, aluminum, aluminum alloys, copper, beryllium oxide, diamond, aluminum nitride, or one or more combinations thereof.

In yet another embodiment, the plurality of thermally conductive layers comprises tellurium copper rod, aluminum, magnesium and aluminum alloys, copper, polymers, ceramics, fibers, beryllium oxide, diamond, aluminum nitride, or one or more combinations thereof. In one embodiment, the cooling device further comprises a heat sink selected from a water-copper heat pipe, and a fan for removing accumulated heat in the thermoelectric assembly.

In still one embodiment, the present invention is directed to an extra-oral thermoelectric anesthetic cooling device comprising a device body comprising a handle; a distal head end, wherein the distal head end comprises a head section and a neck section; a proximal end; and a plurality of thermally conductive layers for dissipating heat from the cooling device; and wherein the body comprises a continuous body, and wherein the handle, the head section and the distal head end comprise a single piece of material. In one embodiment, the cooling device further comprises a thermoelectric assembly, wherein the thermoelectric assembly comprises a plurality of thermoelectric plates, wherein the plurality of thermoelectric plates comprises at least one inner thermoelectric plate; at least one outer thermoelectric plate; and a layer of semiconductor material arranged with spaced apart portions of P-type semiconductor and N-type semiconductor material, wherein the layer is disposed between the at least one inner thermoelectric plate and the at least one outer thermoelectric plate, wherein the at least one inner thermoelectric plate provides cooling; the at least one inner thermoelectric plate is adjacent to a first thermally conductive layer; and wherein a user contacts the patient's target skin tissue through a cool surface at the head section of the device.

In one embodiment, the thermoelectric assembly is configured to cool a patient's skin target surface; and the cooling device further comprises an electronic assembly configured to energize the thermoelectric assembly, wherein the electronic assembly is electrically interconnected to the thermoelectric assembly, wherein the handle is configured to support or contain the thermoelectric assembly.

In one embodiment, the plurality of thermoelectric plates comprises ceramic, glass, porcelain or composite polymer, and the thermoelectric plates are cascaded, are in a multi-stage thermoelectric coolers, or are stacked on top of each other.

In yet another embodiment, the heat generated during operation of the plurality of thermoelectric plates is dissipated by a second thermally conductive layer located on the exterior of the distal head end and the exterior of the handle. In one embodiment, the first thermally conductive layer comprises copper, aluminum alloys, water-copper heat pipe, or copper heat pipe. In another embodiment, the second thermally conductive layer comprises tellurium copper rod, aluminum, copper, aluminum alloys, beryllium oxide, diamond, aluminum nitride, or one or more combinations thereof.

In one embodiment, the plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

In one embodiment, the cooling device further comprises a heat sink selected from a water-copper heat pipe, and a fan for removing accumulated heat in the thermoelectric assembly.

These and other benefits, advantages and features of the present invention will become more full apparent from the following description and appended claims, or may be learned by the practice of the invention as set forth hereinafter.

### BRIEF DESCRIPTION OF THE DRAWINGS

Non-limiting and non-exhaustive embodiments of the present embodiments are described with reference to the following figures, wherein like reference numerals refer to like parts throughout the various views unless otherwise specified.

FIG. 1 depicts a top perspective view of an intra-oral thermoelectric anesthetic cooling device including a device body comprising a proximal gripping end and a distal head end with the thermoelectric assembly in the head, temperature controller, and power supply within the body according to an exemplary embodiment of the present invention.

FIG. 2 depicts a bottom perspective view of an intra-oral thermoelectric anesthetic cooling device with the thermoelectric assembly in the head, temperature controller, and power supply within the body according to an exemplary embodiment of the present invention.

FIG. 3 depicts a cross-sectional view of an intra-oral thermoelectric anesthetic cooling device with the thermoelectric assembly in the head, temperature controller, and power supply within the body according to an exemplary embodiment of the present invention.

FIG. 4 depicts a cross-sectional view of an intra-oral thermoelectric anesthetic cooling device with the thermoelectric assembly, temperature controller, and power supply within the body according to an exemplary embodiment of the present invention.

FIG. 5 depicts a cross-sectional view of an intra-oral thermoelectric anesthetic cooling device with a cavity within the body according to an exemplary embodiment of the present invention.

FIG. 6 depicts a cross-sectional view of the distal head portion of the intra-oral thermoelectric anesthetic cooling device with the thermoelectric assembly within the distal head according to an exemplary embodiment of the present invention.

FIG. 7 depicts a cross-sectional view of the distal head portion of the intra-oral thermoelectric anesthetic cooling device with the thermoelectric assembly within the handle according to an exemplary embodiment of the present invention.

FIG. 8 depicts a perspective view of an extra-oral thermoelectric anesthetic cooling device including a device body comprising a proximal gripping end and a distal head end with the thermoelectric assembly, temperature controller, and power supply within the body according to an exemplary embodiment of the present invention.

FIG. 9 depicts a bottom perspective view of an extra-oral thermoelectric anesthetic cooling device with the thermoelectric assembly in the head, temperature controller, and power supply within the body according to an exemplary embodiment of the present invention.

FIG. 10 depicts a cross-sectional view of an extra-oral thermoelectric anesthetic cooling device with the thermoelectric assembly, temperature controller, and power supply within the body according to an exemplary embodiment of the present invention.

FIG. 12 depicts a cross-sectional view of an extra-oral thermoelectric anesthetic cooling device with a cavity within the body according to an exemplary embodiment of the present invention.

FIG. 13 depicts a cross-sectional view of an extra-oral thermoelectric anesthetic cooling device with the thermoelectric assembly in the handle, temperature controller, and power supply within the body according to an exemplary embodiment of the present invention.

FIG. 14 depicts a cross-sectional view of an extra-oral thermoelectric anesthetic cooling device with the thermoelectric assembly within the distal head end, temperature controller, and power supply within the body according to an exemplary embodiment of the present invention.

### DETAILED DESCRIPTION OF THE INVENTION

The intra-oral and extra-oral thermoelectric anesthetic cooling devices of the present invention provide transient nerve cooling block of the peripheral nerves system. The thermoelectric cooling devices of the present invention allow for temperature controlled cooling of the skin/epidermis or mucous membrane/mucosa, to alleviate pain associated with medical treatment such as injections and skin ablation applied to the human body. The thermoelectric anesthetic cooling devices of the present invention provide a novel method to provide pain relief using thermoelectric cooling for medical procedures, such as pre-anesthetic and post-anesthetic therapy. This pre-anesthetic, post-anesthetic therapy cooling device takes advantage of the Peltier effect to create a heat flux between the junction of two different types of materials. The Peltier effect is the presence of heating or cooling at an electrified junction of two different conductors. The Peltier cooler is a solid-state active heat pump, which transfers heat from one side to the other, with consumption of electrical energy, depending on the direction of the current.

In general, a thermoelectric cooler (TEC) comprises two sides, and when DC electricity flows through the device (i.e., the device is energized), it brings heat from one side to the other, so that one side becomes cooler while the other side becomes hotter. The “hot” side is coupled to the head of the thermoelectric anesthetic cooling device. The neck and body of the device is used as a heat sink so that it remains at ambient temperature, while the cool side temperature is reduced below room temperature. The “cool” side can be placed against the epidermis or mucosa to achieve a transient conduction block in the peripheral nerves that does not result in onset firing. Partial conduction block is found for temperatures below 14° C., and complete nerve conduction block is achieved for temperatures below 6° C. This provides an analgesic effect to reduce or prevent pain from injections and inflammation. Also, the cooling device minimizes bruising associated with injectable fillers, and pain from a variety of laser treatments.

Two unique semiconductors, one N-type and one P-type, are used because they need to have different electron densities. The semiconductors are placed thermally in parallel to each other, and electrically in series, then joined with a thermally conducting plate on each side. When a voltage is applied to the free ends of the two semiconductors, there is a flow of DC current across the junction of the semiconductors, thereby causing a temperature difference.

A TEC device does not contain any moving parts so maintenance is required less frequently, while temperature control within fractions of a degree can be maintained. Furthermore, a TEC device has a flexible shape (form factor), and in particular, it can have a very small size, having a long life with mean time between failures (MTBF) exceeding 100,000 hours, and is controllable via changing the input voltage/current.

The following detailed description is merely exemplary in nature and is not intended to limit the invention or the application and uses of the invention. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the invention and not to limit the scope of the invention that is defined by the claims. Furthermore, there is no intention to be bound by any expressed or implied theory presented in the preceding technical field, background, brief summary, or the following detailed description.

As used herein, and unless the context dictates otherwise, the term “thermoelectric cooling device” is intended to include an intra-oral thermoelectric anesthetic cooling device, cooling device, and device. Therefore, the terms “thermoelectric cooling device”, “intra-oral thermoelectric anesthetic cooling device”, “cooling device” and “device”, may be used interchangeably.

As used herein, and unless the context dictates otherwise, the term “thermoelectric cooling device” is intended to include an extra-oral thermoelectric anesthetic cooling device, cooling device, and device. Therefore, the terms “thermoelectric cooling device”, “extra-oral thermoelectric anesthetic cooling device”, “cooling device”, and “device”, may be used interchangeably.

As used herein, and unless the context dictates otherwise, the term “switch” is intended to include button. Therefore, the terms “switch”, and “button”, may be used interchangeably.

As used herein, and unless the context dictates otherwise, the term “device body” is intended to include body. Therefore, the terms “device body”, and “body”, may be used interchangeably.

As used herein, and unless the context dictates otherwise, the term “thermoelectric assembly” is intended to include thermoelectric cooler. Therefore, the terms “thermoelectric assembly”, and “thermoelectric cooler”, may be used interchangeably.

As used herein, and unless the context dictates otherwise, the term “handle” is intended to include gripping portion. Therefore, the terms “handle”, and “gripping portion”, may be used interchangeably.

In this description, reference is made to the drawings, wherein like parts are designated with like reference numerals throughout. As used in the description herein and throughout, the meaning of “a,” “an,” and “said” includes plural reference unless the context clearly dictates otherwise. Also, as used in the description herein, the meaning of “in” includes “into” and “on”, unless the context clearly dictates otherwise.

As used herein, the term “about” in conjunction with a numeral refers to a range of that numeral starting from 10% below the absolute of the numeral to 10% above the absolute of the numeral, inclusive.

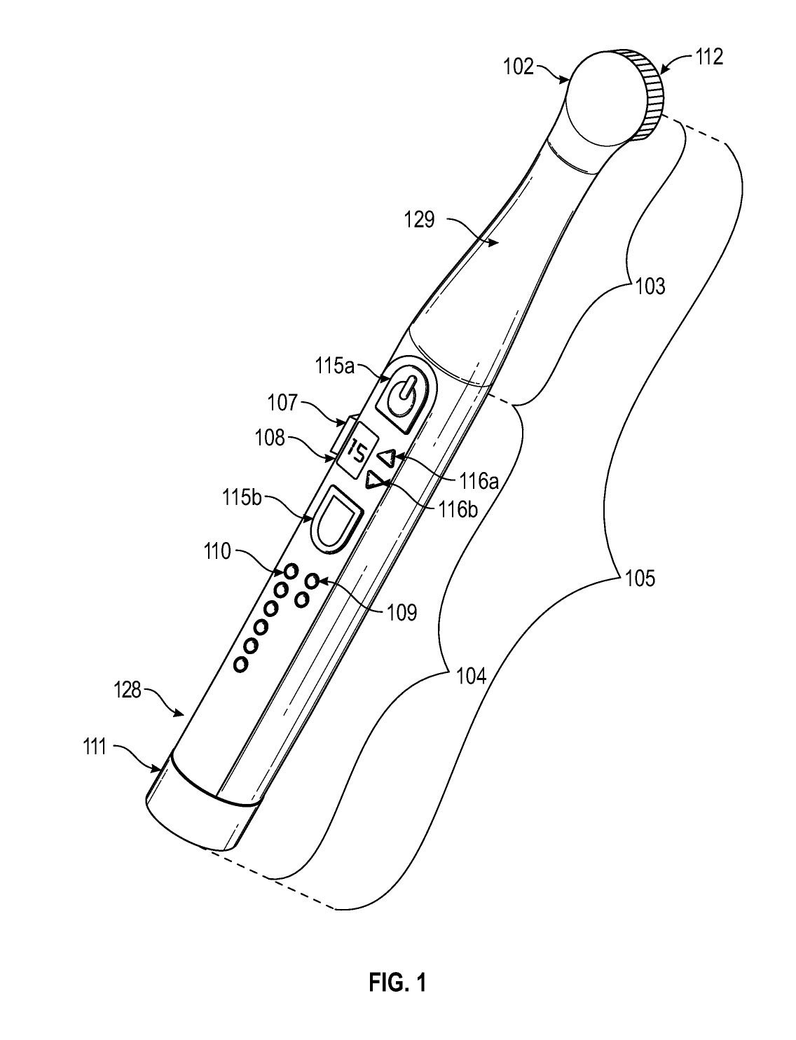

An exemplary configuration of the thermoelectric cooling device is schematically depicted in FIG. 1, in which an intra-oral thermoelectric anesthetic cooling device 105 comprises body 128 including handle 104, distal head end 103, and proximal end 111. Distal head end 103 comprises head section 102, and a neck section 129. Distal head end 103 is sized and configured to be inserted into the mouth of a patient. In one embodiment, head section 102 comprises a thermoelectric assembly 112 that allows a user to cool a patient's intra oral tissue. In another embodiment, thermoelectric assembly 112 comprises thermoconductive coating for hygienic purposes. In an alternative embodiment, head section 102 comprises a thermoelectric assembly of thermoelectric coolers 112 stacked. In one embodiment, handle 104 comprises a thickness of about 15 to about 50 mm, and in one or more embodiments, a thickness of about 14 to about 35 mm. In general, dimensions for handle 104 are used that provide comfort for a user (i.e., a clinician). In one embodiment, neck section 129 and head section 102 are thinner than handle 104 and comprises a thickness of about 1 to about 30 mm. In one embodiment, head section 102 that comes interfaces patient's mucosa comprises a diameter of about 2 to about 20 mm, neck section 129 comprises a diameter of about 60 mm to 80 mm; and handle 104 is about 10 to about 30 mm wide and is about 150 to about 250 mm long.

In one or more embodiments, device body 128 comprises a suitable thermally conductive material, including but not limited to, thermally conductive metals (e.g., aluminum, copper, magnesium and aluminum alloy), fibers and/nanomaterials, ceramics and polymers.

In one embodiment, all or a portion of the exterior surface of device body 128 includes one or more coatings to protect the surface and/or to facilitate cleaning and/or sterilizing of the cooling device 105 (not shown). In one embodiment, fluoropolymer coatings such as Polytetrafluorethylene (PTFE) may be used due to the non-sticky properties. In another embodiment, the exterior surface of device body 128 is coated with a scratch resistant coating (not shown). The coating layers may be applied by chemical or plasma vapor operation, or other techniques known in the art. In one embodiment, scratch resistant coating comprises metal oxide or metal nitride or the same as the thermally conductive layer.

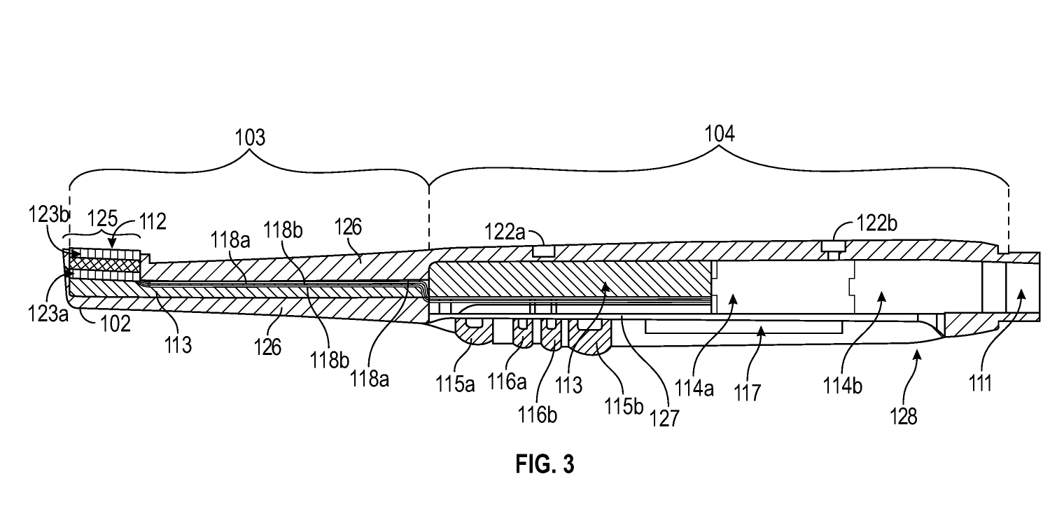

In an embodiment, body 128 comprises temperature controller 116 including buttons 116a and 116b, temperature display 108, power button 115a, optional vibration switch 107, timer/mode button 115b, warning indicator lights 109, and timing indicator lights 110. In one embodiment, proximal end 111 comprises a screwable lid for inserting batteries 114a and 114b (as shown in FIG. 3). In one embodiment, temperature controller 116 allows a temperature range from about 21° C. to about 0° C. to eliminate the risk of frostbite on the patient's tissue. In one exemplary embodiment, as depicted in FIG. 1, buttons 116a and 116b increase and decrease, respectively, the temperature according to the user's need.

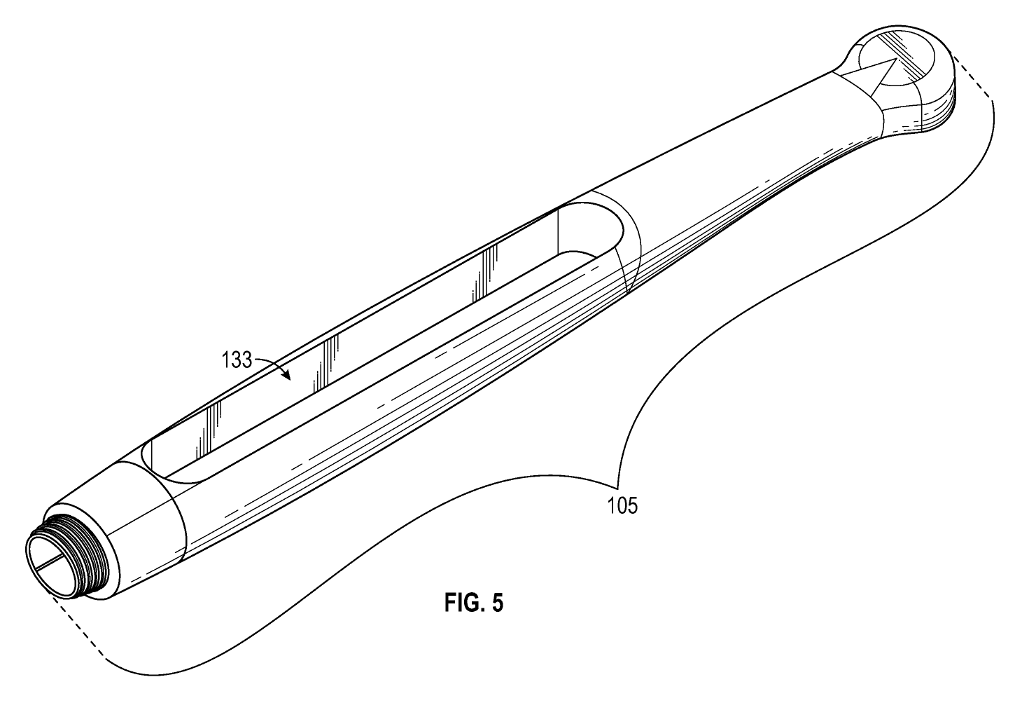

In one embodiment, thermoelectric anesthetic cooling device 105 comprises an electronic assembly 117 located within a cavity 133 of body 128 (as shown in FIG. 5). In one embodiment, cavity 133 comprises a rim (not shown) that is configured to form a secure fit with a corresponding rim of electronic assembly 117 (not shown) to ensure proper sealing of cavity 133. Electronic assembly 117 comprises batteries 114a and 114b, circuit board 127, and power button 115a. In this exemplary embodiment, electronic assembly 117 is used to energize thermoelectric cooling device 105. Electronic assembly 117 allows a user (i.e., a clinician) to turn on/off device 105 by using power button 115a. In an alternative embodiment, power can be provided by a power cord (not shown) that is coupled to electronic assembly 117, located in proximal end 111. In an embodiment, button 115b provides a user options for different mode and time for operating device 105. In one embodiment, thermoelectric cooling device 105 comprises batteries 114a and 114b with or without the ability to adapt to a charging station, or a cord connected to a power outlet with different adapters. In one embodiment, rechargeable batteries 114a and 114b provide power to electronic assembly 117. In one or more exemplary embodiments, holes 122a and 122b allow electronic assembly 117 to be secured to body 128 by using screws (see FIG. 3).

Circuit board 127, power button 115a, power source (such as batteries 114a and 114b, internal or external power source, plug), electronic assembly 117 and thermoelectric assembly 112 are all electronically interconnected.

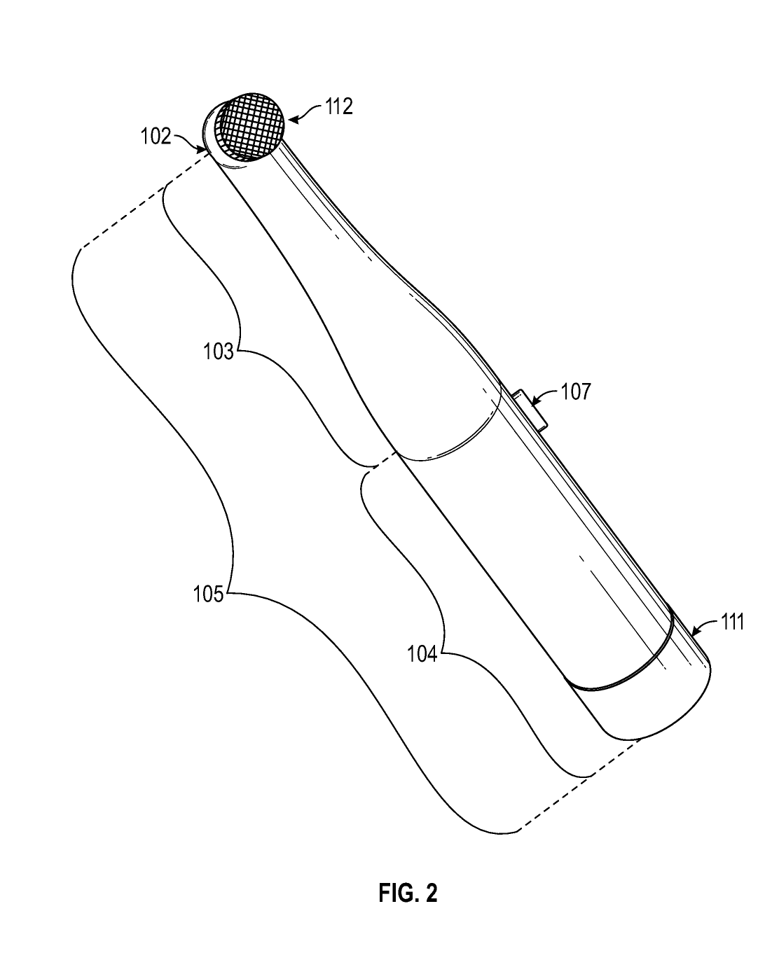

FIG. 2 is an exemplary embodiment of the posterior view of intra-oral device 105 depicting thermoelectric cooling plates of thermoelectric assembly 112, distal head end 103, head section 102, optional vibration switch 107, handle 104, and proximal end 111. In an embodiment, head section 102 supports or contains thermoelectric assembly 112. In one embodiment, optional vibration switch 107 provides additional pain reduction for the patient's target tissue.

In one exemplary embodiment of the present invention, the power input and thermoelectric output of the thermoelectric assembly 112 are ramped up over a period of time. In another exemplary embodiment, temperature controller 116 is designed to provide many possible ramp-up times and temperature ranges (e.g., about 15° C. to about 0° C.). In one or more exemplary embodiments, a ramp-up time may be appropriate for one scenario, but not for another. In one embodiment, thermoelectric cooling device 105 may include circuitry configured to allow a user to choose a ramp time for ramping-down the temperature of the thermoelectric cooling device (not shown).

In one embodiment, electronics assembly 117 comprises a plurality of selectable ramp-up times within a range from about 5 seconds to about 10 minutes. In this embodiment, a user selects one of the plurality of ramp-up times and thermoelectric cooling device 105 incrementally increases power input to reach the selected temperature in the selected period of time (not shown).

In one exemplary embodiment, body 128 comprises a continuous body, also known as a unibody, wherein handle 104, head section 102 and distal head end 103 comprise a single piece of material. In one embodiment, body 128 comprises one or more thermally conductive body materials (e.g., thermally conductive metal, polymer, ceramic, and/or thermally conductive ceramic fibers or nanomaterials). In this embodiment, the unibody provides a seamless body 128 and maximizes heat conduction into body 128.

In one embodiment, thermoelectric cooling device 105 comprises an elongated shaped body to facilitate use of cooling device 105 in the mouth of a patient. One of ordinary skill in the art will appreciate that thermoelectric cooling device 105 may have other shapes suitable for use in cooling the patient's epidermis/mucosa within, or even outside, a patient's mouth. For example, thermoelectric anesthetic cooling device 105 having a gun-like configuration may incorporate any of the features disclosed herein. In general, any contra-angled configuration may be used in connection with the features described herein. In one or more embodiments, thermoelectric anesthetic cooling device 105 may have a wire that it is directly compatible with standard power outlets (including all relevant male adapters for ex-US use), a battery pack (i.e., 114a and 114b) that is housed inside proximal end 111 of handle 104, or the thermoelectric anesthetic cooling device may have a rechargeable docking station.

In another embodiment, thermoelectric anesthetic cooling device 105 comprises a shape suitable for use in an intra-orally or extra-orally cooling device. In an embodiment, thermoelectric plates 123 comprise different sizes, shapes (square or round) depending on the use. In one or more embodiments, multiple adaptors are used that reside on the head of thermoelectric plates 123 (not shown). The adaptors may comprise metal or other appropriate substance for better contact with the mucosa/epidermis.

In yet another embodiment, the entirety or a portion of body 128 is produced from a thermally conductive body material so long as device body 128 has sufficient thermal conductivity to dissipate the desired heat generated by the thermoelectric unit during use (i.e., with the device set to a maximum user selectable coldest temperature output). In this embodiment, device body 128 provides heat dissipation, thus the need for a configuration to accommodate a separate heat sink is eliminated for thermoelectric anesthetic cooling device 105.

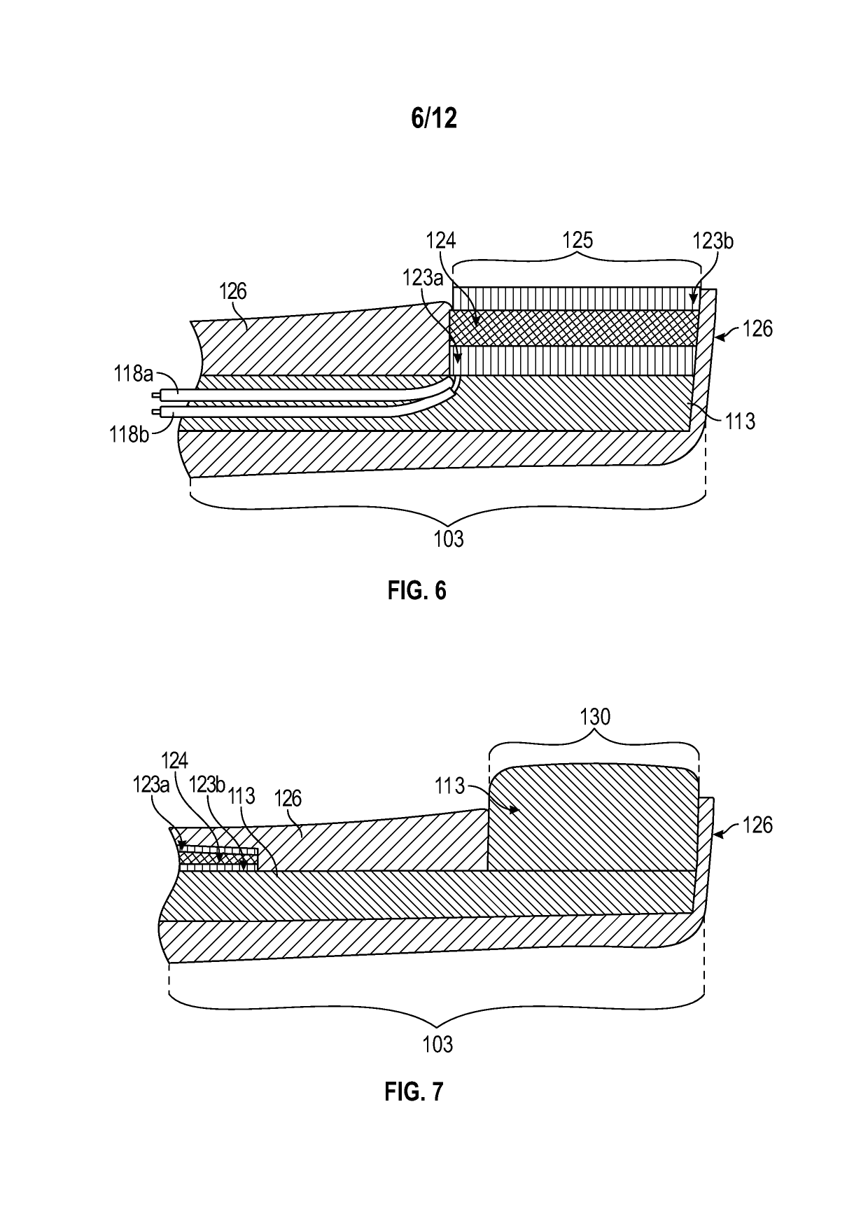

In an exemplary embodiment, as depicted in FIGS. 3 and 6, thermoelectric assembly 112 resides within distal head end 103, in particular in head section 102. In this embodiment, thermoelectric assembly 112 comprises inner ceramic plate 123a and outer ceramic plate 123b, and layer 124 (as shown in FIG. 6) of semiconductor material arranged with spaced apart portions of P-type semiconductor and N-type semiconductor material, wherein layer 124 is disposed between inner ceramic plate 123a and outer ceramic plate 123b. In one or more embodiments, ceramic plates 123a and 123b comprise glass, porcelain or composite polymer. In an alternative embodiment, thermoelectric assembly 112 comprises a plurality of thermoelectric plates, such as ceramic plates that are cascaded, are in a multi-stage thermoelectric cooler, or are stacked on top of each other (not shown). In one embodiment, power supplied by rechargeable batteries 114a and 114b, through wires 118a and 118b located in electronic assembly 117, charge carriers and flow to layer 124, giving rise to the Peltier effect, such that cooling is provided at outer ceramic plate 123b and heating is provided at inner ceramic plate 123a. A user (i.e., clinician) interfaces target tissue on the patient's skin through outer ceramic plate 123b through cold surface 125, as depicted in FIG. 6. In an embodiment, thermoelectric assembly 112 comprises a temperature sensor attached to outer ceramic plate 123b located on head section 102 electrically coupled to the terminal block (not shown). The heat generated in inner ceramic plate 123a is dissipated from thermoelectric assembly 112 using first thermally conductive layer 113 located within interior of body 128 (heat sink), and second thermally conductive layer located on the exterior of distal head end 103 and the exterior of handle 104. In one embodiment, the peripheral nerve in the skin/mucosa is cooled from normal body temperature (about 37° C.) down to about 5° C.

In one or more exemplary embodiments, first thermally conductive layer 113 comprises materials with high thermal conductivity such as copper, aluminum alloys, and copper heat pipe with or without water. In one embodiment, first thermally conductive layer 113 comprises a flat water-copper heat pipe, which allows a smaller head section 102, while keeping a low delta T in thermoelectric assembly 112. This embodiment provides the thermal mass to absorb the heat transferred from the target's tissue as well as the heat generated by the operation of thermoelectric assembly 112 during a 2 minute treatment without an excessive temperature rise in thermoelectric anesthetic cooling device 105. In this embodiment, a cylindrical heat sink is used as second thermally conductive layer 126. Second thermally conductive layer 126 comprises a 19 mm tellurium copper rod. Inner ceramic plate 123a is attached to a flat water-copper heat pipe using a semi-flexible silver-filled epoxy to allow different materials to contract at different rates without inducing excessive stress. In one or more exemplary embodiments, second thermally conductive layer 126 comprises materials with high thermal conductivity such as aluminum, copper, or aluminum alloys plus an additional layer of conductive material. In these embodiments, the surface area of second thermally conductive layer 126 is sufficiently large such that a majority (e.g., substantially all) of the heat conducted away from the thermoelectric assembly 112 by second thermally conductive layer 126 is transferred to device body 128.

In one or more embodiments, second thermally conductive layer 126 may comprise a separate piece that is secured to a portion of device body 128 and may have a thickness ranging from about 100 microns to about 1.5 mm and is produced from one or more highly thermally conductive materials such as, but not limited to, beryllium oxide, diamond, aluminum nitride, or one or more combinations of these materials.

In another embodiment, second thermally conductive layer 126 may comprise a very thin layer applied over at least a portion of device body 128 (e.g., by chemical or plasma vapor deposition or plasma flame spraying). In such an embodiment, the thickness of second thermally conductive layer 126 may be only about 0.05 micron to about 50 microns. The thickness and surface area of second thermally conductive layer 126 is sufficient to ensure that most, if not essentially all, of the waste heat generated by thermoelectric assembly 112 is transferred through thermally conductive layer 126 and dissipated into body 128 material. At moderate to low operating temperatures, second thermally conductive layer 126 can dissipate heat from the substrate of thermoelectric assembly at the same rate that heat is dissipated into the thermoelectric assembly 112 substrate from thermoelectric assembly 112 thereby allowing continuous moderate to low temperature operation.

In another embodiment, intra-oral thermoelectric cooling device 105 may contain water or a fan as a heat sink to remove accumulated heat in thermoelectric assembly 112.

In one embodiment, thermoelectric assembly 112 comprises different sized thermoelectric plates, types (e.g., high performance, micro, multi-stage, series-parallel, and standard) and shapes, and a thermally conductive layer (not shown). In another embodiment, thermally conductive layer may comprise a separate, relatively thick member that is secured to body, rather than being a very thin layer applied by vapor deposition or plasma flame spraying techniques. In a further embodiment, a relatively thin thermally conductive layer is applied to cooling device 105 of the intra-oral thermoelectric anesthetic cooling device (e.g., by vapor deposition or plasma flame spraying).

In one or more embodiments, the exterior surface of cooling device 105 may be coated with one or more coatings to protect the surface and/or facilitate cleaning and/or sterilizing thermoelectric anesthetic cooling device 105. In one or more embodiments, a thermally conductive grease, gel, or adhesive can include a filler material to improve thermal conductivity.

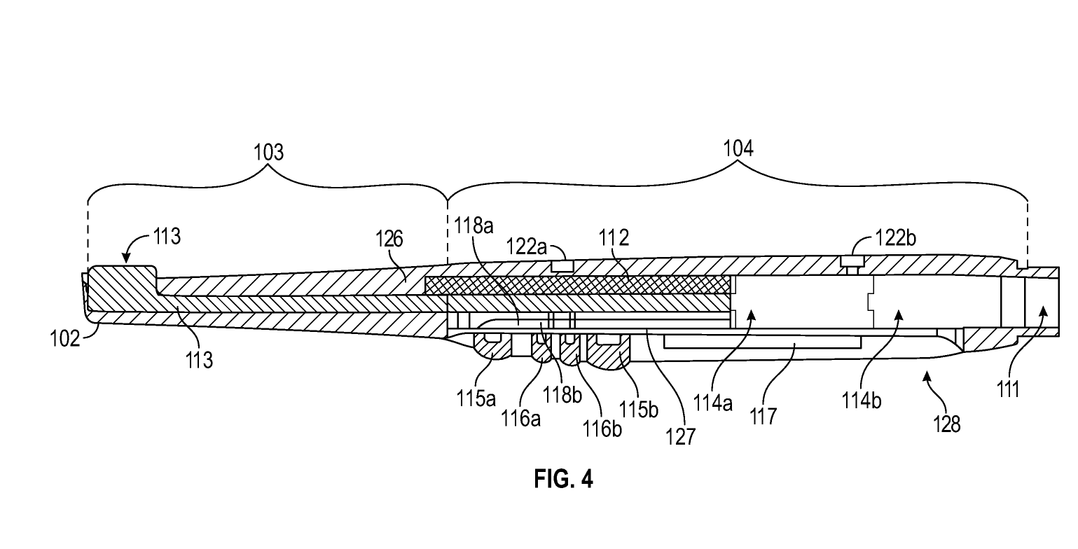

In an exemplary embodiment, as depicted in FIGS. 4, and 7, thermoelectric assembly 112 resides within body 128. In this embodiment, thermoelectric assembly 112 resides in handle 104. In this embodiment, thermoelectric assembly 112 comprises ceramic plates 123a and 123b, and layer 124 (as shown in FIG. 4) of semiconductor material arranged with spaced apart portions of P-type semiconductor and N-type semiconductor material, wherein layer 124 is disposed between ceramic plates 123a and 123b. In an alternative embodiment, thermoelectric assembly 112 comprises a plurality of thermoelectric plates, such as ceramic plates that are cascaded, are in a multi-stage thermoelectric cooler, or are stacked on top of each other (not shown). In one embodiment, power supplied by rechargeable batteries 114a and 114b, through wires 118a and 118b located in electronic assembly 117, charge carriers and flow to layer 124, giving rise to the Peltier effect, such that cooling is provided at inner ceramic plate 123b and heating is provided at outer ceramic plate 123a. In this embodiment, first thermally conductive layer 113 is located in head section 102 and extends through part of handle 104, as shown in FIG. 4. Furthermore, first thermally conductive layer 113 is adjacent to inner ceramic plate 123b. In this embodiment, cooling of inner ceramic plate 123b is conducted through first thermally conductive layer 113 to head section 102. A user (i.e., clinician) interfaces target tissue on the patient's mucosa through cold surface 130, as depicted in FIG. 7. In an embodiment, thermoelectric assembly 112 comprises a temperature sensor attached to inner ceramic plate 123b and electrically coupled to the terminal block (not shown). The heat generated in outer ceramic plate 123a is dissipated from thermoelectric assembly 112 using second thermally conductive layer 126 located on the exterior of distal head end 103 and the exterior of handle 104. In one embodiment, the peripheral nerve in the skin/mucosa is cooled from normal body temperature (about 37° C.) down to about 5° C.

In an alternative embodiment, thermoelectric assembly 112 comprises 2 sets of ceramic plates 123a and 123b, located above first thermally conductive layer 113 and below first thermally conductive layer 113 (not shown). In another embodiment, thermoelectric assembly 112 comprises one set of ceramic plates 123a and 123b located either above first thermally conductive layer 113 or below first thermally conductive layer 113 (not shown).

In one or more exemplary embodiments, first thermally conductive layer 113 comprises copper, aluminum alloys, and copper heat pipe with or without water. In one or more exemplary embodiments, second thermally conductive layer 126 comprises aluminum, copper, aluminum alloys plus an additional layer of conductive material. In these embodiments, the surface area of second thermally conductive layer 126 is sufficiently large that a majority (e.g., substantially all) of the heat conducted away from the thermoelectric assembly 112 by second thermally conductive layer 126 is transferred to device body 128.

In another embodiment, thermoelectric cooling device may contain water or a fan as a heat sink to remove accumulated heat in thermoelectric assembly 112.

The present invention also provides a method of selective localized cooling of patient's target tissue by using intra-oral thermoelectric cooling device 105. In an exemplary embodiment, batteries 114a and 114b of intra-oral thermoelectric cooling device 105 are charged by using the plug charger into the electrical outlet or placing thermoelectric cooling device 105 onto a charging dock station which is connected to an outlet. A single use hygienic barrier sleeve is placed onto head section 102 of cooling device 105. In an embodiment, where thermoelectric assembly 112 is within distal head end 103, head section 102 is then placed onto the target tissue (i.e., the intra-oral tissue of patient's skin) that needs to be cooled (i.e., anesthetized). Power button 115a is then turned on to start thermoelectric cooling device 105. Optional vibration may used at this point by pressing vibration switch 107. Desired temperature is reached by using 116a (to increase the temperature) or by using 116b (to decrease the temperature). The operation time is set by using time/mode button 115b. Timing indicator light 110 turns green to show desired time interval. Thermoelectric cooling device 105 operates at 45-second bursts and not continuously. Button 115b shows the power level; an orange light in warning indicator lights 109 indicates a low battery level and a red light indicates thermoelectric cooling device 105 needs to be cooled.

The current provided by the batteries 114a and 114b in the electronic assembly 117 travels through device 105 and is transferred to the thermoelectric assembly 112 by wires 118a and 118b, causing outer ceramic thermoelectric plate 123b to cool down to 4° C. (or other desired temperature as indicated on temperature display 108). The user (i.e., clinician) places cold surface (i.e., 125) of outer ceramic plate 123b in contact with skin surface of the target tissue, causing the target tissue to become numb. The aforementioned method is repeated as needed for any site in the mouth of the patient. Power button 115a is pressed to turn intra-oral thermoelectric cooling device 105 off. The hygienic sleeve is then removed and cooling device 105 is wiped down with sanitizing solution before applying the device to the next patient.

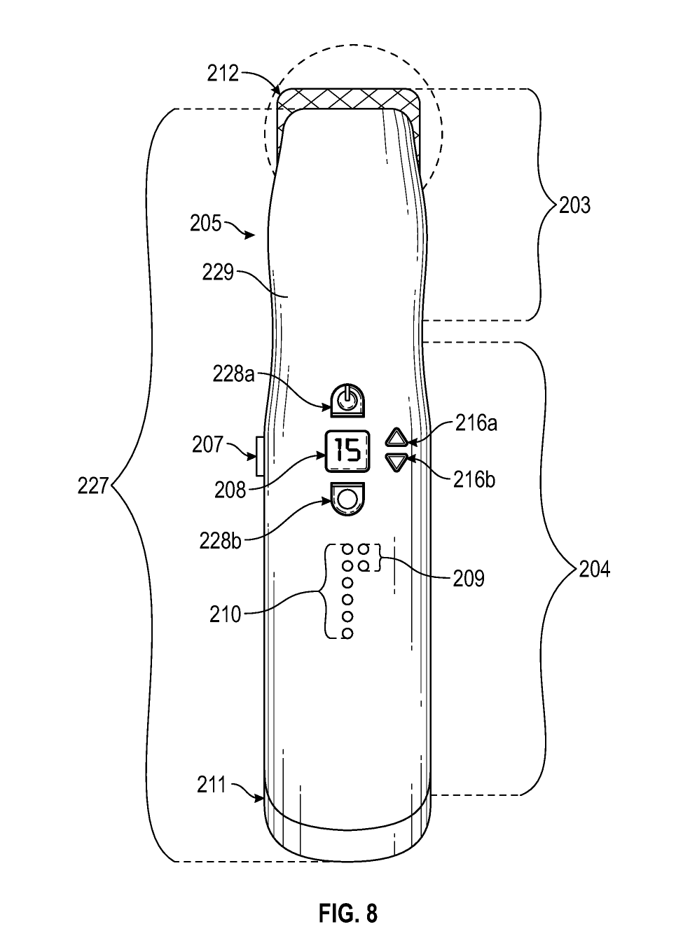

An exemplary configuration of the thermoelectric cooling device is schematically depicted in FIG. 8, in which an extra-oral thermoelectric anesthetic cooling device 227 comprises body 205 including handle 204, distal head end 203, and proximal end 211. distal head end 203 comprises head section 202, and neck section 229. In one embodiment, head section 202 comprises an thermoelectric cooling assembly of thermoelectric cooler 212 that allows a user to cool a patient's skin surface. In another embodiment, thermoelectric cooler 212 comprises thermoconductive coating for hygienic purposes. In an alternative embodiment, head section 202 comprises an thermoelectric cooling assembly of thermoelectric coolers 112 stacked. In one embodiment, handle 204 comprises a thickness of about 15 to about 50 mm, and in one or more embodiments, a thickness of about 14 to about 35 mm. In general, dimensions for handle 204 are used that provide comfort for a user (i.e., a clinician). In one embodiment, neck section 229 and head section 202 are thinner than handle 204 and comprises thickness of about 20 to about 35 mm. In one embodiment, head 202 that interfaces with patient's epidermis is about 10 mm to about 50 mm, neck section 229 is about 10 to about 60 mm; and handle 204 is about 100 to about 120 mm long, is about 20 to about 35 mm wide. In another embodiment, handle 240 is about 150 to about 180 mm long.

In one or more embodiments, device body 205 comprises a suitable thermally conductive material, including but not limited to, thermally conductive metals (e.g., aluminum, copper, magnesium and aluminum alloy), fibers and/nanomaterials, ceramics and polymers.

In one embodiment, all or a portion of the exterior surface of device body 205 includes one or more coatings to protect the surface and/or to facilitate cleaning and/or sterilizing of the extra oral cooling device 227 (not shown). In one embodiment, fluoropolymer coatings such as Polytetrafluorethylene (PTFE) may be used due to the non-sticky properties. In another embodiment, the exterior surface of device body 205 is coated with a scratch resistant coating (not shown). The coating layers may be applied by chemical or plasma vapor operation, or other techniques known in the art. In one embodiment, scratch resistant coating comprises metal oxide or metal nitride or the same as the thermally conductive layer.

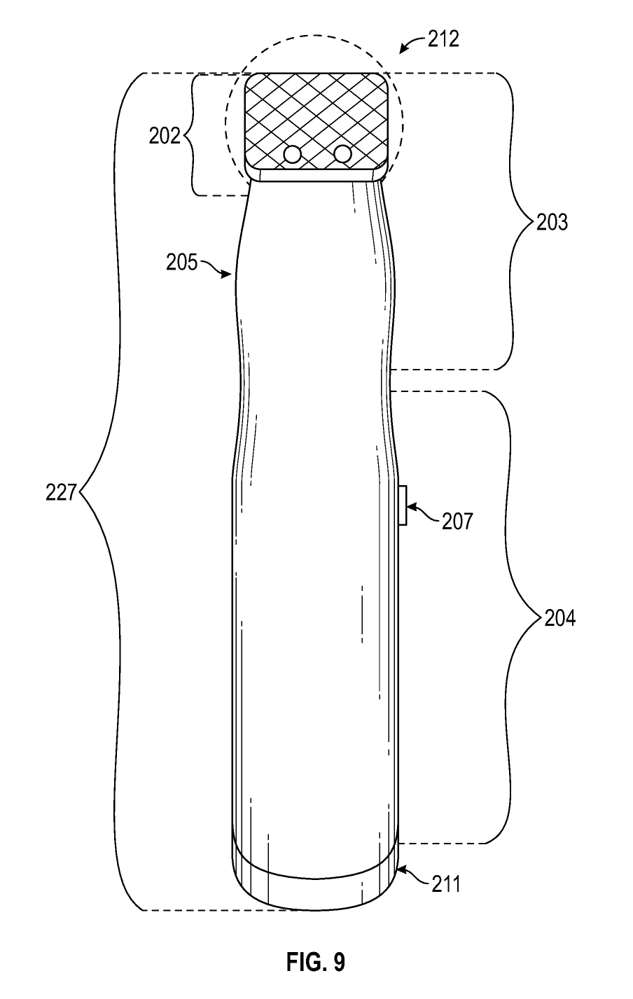

In an embodiment, device body 205 comprises temperature controller 216 including buttons 216a and 216b, temperature display 208, power button 228a, vibration switch 207, timer/mode button 228b, warning indicator lights 209, and timing indicator lights 210. In one embodiment, proximal end 211 comprises a screwable lid for inserting batteries 214a and 214b (as shown in FIG. 9). In one embodiment, temperature controller 216 allows a temperature range from about 21° C. to about 0° C. to eliminate the risk of frostbite on the patient's skin tissue. In one exemplary embodiment, as depicted in FIG. 8, buttons 216a and 216b increase and decrease, respectively, the temperature according to the user's need.

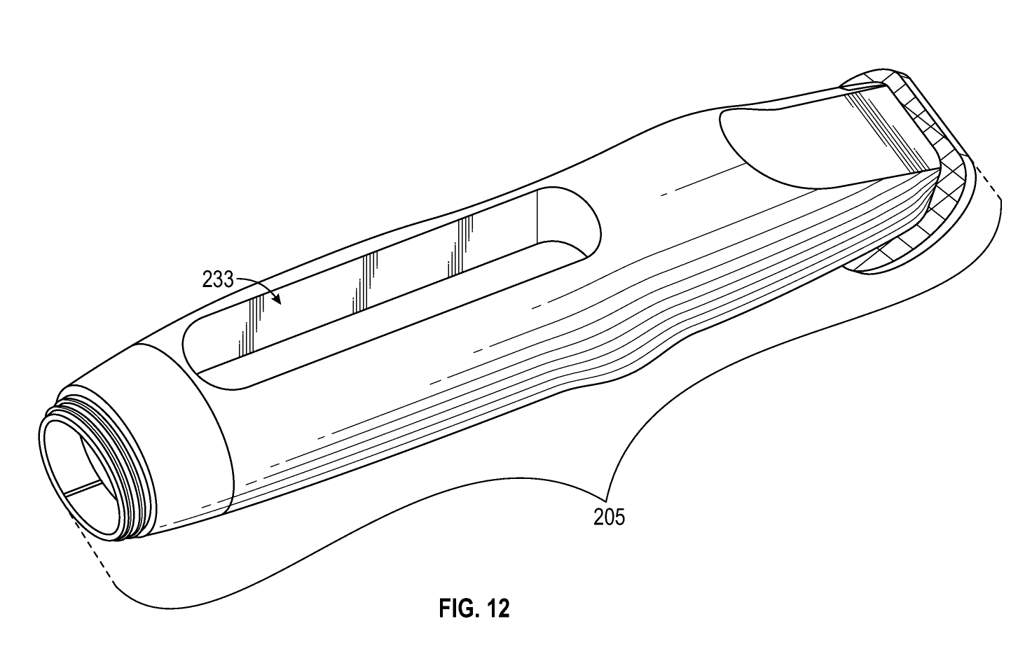

In one embodiment, thermoelectric anesthetic cooling device 227 comprises an electronic assembly 217 located within cavity 233 of body 205 (as shown in FIG. 12). In one embodiment, cavity 233 comprises a rim (not shown) that is configured to form a secure fit with a corresponding rim of electronic assembly 217 (not shown) to ensure proper sealing of cavity 233. In an embodiment, electronic assembly 217 comprises batteries 214a and 214b, circuit board 232, and power button 228a. In this exemplary embodiment, electronic assembly 217 is used to energize extra-oral thermoelectric cooling device 227. Electronic assembly 217 allows a user (i.e., a clinician) to turn on/off cooling device 227 by using power button 215a. In an alternative embodiment, power can be provided by a power cord (not shown) that is coupled to electronic assembly 217, located in proximal end 211. In an embodiment, button 215b provides a user options for different mode and time for operating extra oral cooling device 227.

Circuit board 232, power button 228a, power source (such as batteries 214a and 214b, internal or external power source, plug), electronic assembly 217 and thermoelectric assembly 212 are all electronically interconnected.

In an embodiment, extra oral thermoelectric cooling device 227 comprises batteries 214a and 214b with or without the ability to adapt to a charging station, or a cord connected to a power outlet with different adapters. In one embodiment, rechargeable batteries 214a and 214b provide power to electronic assembly 217.

FIG. 9 is an exemplary embodiment of the posterior view of extra-oral device 227 depicting thermoelectric cooling plates of thermoelectric assembly 212, distal head end 203, head section 202, optional vibration switch 207, handle 204, and proximal end 211. In an embodiment, head section 202 supports or contains thermoelectric assembly 212. In one embodiment, optional vibration switch 207 provides additional pain reduction for the target tissue.

In one exemplary embodiment of the present invention, the power input and thermoelectric output of the thermoelectric assembly 227 are ramped up over a period of time. In another exemplary embodiment, temperature controller 216 is designed to provide many possible ramp-up times and temperature ranges. In one embodiment, the temperature ranges from about 15 to about 0. In one or more exemplary embodiments, a ramp-up time may be appropriate for one scenario, but not for another. In one embodiment, thermoelectric cooling device 227 may include circuitry configured to allow a user to choose a ramp time for ramping-down the temperature of the thermoelectric cooling device (not shown).

In one embodiment, electronics assembly 217 comprises a plurality of selectable ramp-up times within a range from about 5 seconds to about 10 minutes. In this embodiment, a user selects one of the plurality of ramp-up times and thermoelectric cooling device 227 incrementally increases power input to reach the selected temperature in the selected period of time (not shown).

In one exemplary embodiment, device body 205 comprises a continuous body, also known as a unibody, wherein handle 204, head section 202 and distal head end 203 comprise a single piece of material. In one embodiment, body 205 comprises one or more thermally conductive body materials (e.g., thermally conductive metal, polymer, ceramic, and/or thermally conductive ceramic fibers or nanomaterials). In this embodiment, the unibody provides a seamless body 205 and maximizes heat conduction into EX-device body 205.

In one embodiment, thermoelectric cooling device 227 comprises a hand held shaped body to facilitate use of cooling device 227 on the skin of a patient. One of ordinary skill in the art will appreciate that thermoelectric cooling device 227 may have other shapes suitable for use in cooling the patient's epidermis. For example, extra oral thermoelectric anesthetic cooling device 227 having a gun-like configuration may incorporate any of the features disclosed herein. In general, any contra-angled configuration may be used in connection with the features described herein. In one or more embodiments, thermoelectric anesthetic cooling device 227 may have a wire that it is directly compatible with standard power outlets (including all relevant male adapters for ex-US use), an battery pack (i.e., 214a and 214b) that is housed inside proximal end 211 of handle 204, or the thermoelectric anesthetic cooling device may have a rechargeable docking station.

In another embodiment, thermoelectric anesthetic cooling device 227 comprises a shape suitable for use in an or extra-orally cooling device. In an embodiment, thermoelectric plates 223a and 223b comprise different sizes, shapes (square or round) depending on the use. In one or more embodiments, multiple adaptors are used that reside on the head of thermoelectric plates 223a and 223b (not shown). The adaptors may comprise metal or other appropriate substance for better contact with the epidermis.

In yet another embodiment, the entirety or a portion of body 205 is produced from a thermally conductive body material so long as device body 205 has sufficient thermal conductivity to dissipate the desired heat generated by the thermoelectric unit during use (i.e., with the device set to a maximum user selectable coldest temperature output). In this embodiment, device body 205 provides heat dissipation, thus the need for a configuration to accommodate a separate heat sink is eliminated for thermoelectric anesthetic cooling device 227.

In an exemplary embodiment, as depicted in FIGS. 11 and 14, thermoelectric assembly 212 resides within distal head end 203, in particular in head section 202. In this embodiment, thermoelectric assembly 212 comprises inner ceramic plates 223a and outer ceramic plate 223b, and layer 224 of semiconductor material arranged with spaced apart portions of P-type semiconductor and N-type semiconductor material, wherein layer 224 is disposed between ceramic plates 223a and 223b. In one or more exemplary embodiments, ceramic plates 123a and 123b comprise glass, porcelain or composite polymer. In an alternative embodiment, thermoelectric assembly 212 comprises a plurality of thermoelectric plates, such as ceramic plates that are cascaded, are in a multi-stage thermoelectric coolers, or are stacked up on top of each other (not shown). In one embodiment, power supplied by rechargeable batteries 214a and 214b, through wires 218a and 218b located in electronic assembly 217, charge carriers and flow to layer 224, giving rise to the Peltier effect, such that cooling is provided at outer ceramic plate 223b and heating is provided at inner ceramic plate 223a. A user (i.e., clinician) interfaces target tissue on the patient's skin through outer ceramic plate 223b through cold surface 225, as depicted in FIG. 14. In an embodiment, thermoelectric assembly 212 comprises a temperature sensor attached to outer ceramic plate 123b located on head section 102 electrically coupled to the terminal block (not shown). The heat generated in inner ceramic plate 223a is dissipated from thermoelectric assembly 212 using first thermally conductive layer 213 located within interior of body 205, and second thermally conductive layer 215 located on the exterior of distal head end 203 and the exterior of handle 204. In one embodiment, the peripheral nerve in the skin/mucosa is cooled from normal body temperature (about 37° C.) down to about 5° C.

In one or more exemplary embodiments, first thermally conductive layer 213 comprises materials with high thermal conductivity such as copper, aluminum alloys, and copper heat pipe with or without water. In one embodiment, first thermally layer 213 comprises a flat water-copper heat pipe, which allows a smaller head section 202, while keeping a low delta T in thermoelectric assembly 212. This embodiment provides the thermal mass to absorb the heat transferred from the target's tissue as well as the heat generated by the operation of thermoelectric assembly 212 during a 2 minute treatment without an excessive temperature rise in extra oral thermoelectric anesthetic cooling device 227. In this embodiment, a cylindrical heat sink is used as second outer thermally conductive layer 215. Second thermally conductive layer 215 comprises a 19 mm tellurium copper rod. Inner ceramic plate 223a is attached to a flat water-copper heat pipe using a semi-flexible silver-filled epoxy to allow different materials to contract at different rates without inducing excessive stress. In one or more exemplary embodiments, second thermally conductive layer 215 comprises materials with high thermal conductivity such as aluminum, copper, or aluminum alloys plus an additional layer of conductive material. In these embodiments, the surface area of second thermally conductive layer 215 is sufficiently large such that a majority (e.g., substantially all) of the heat conducted away from thermoelectric assembly 212 by second thermally conductive layer 215 is transferred to device body 205.

In one or more embodiments, second thermally conductive layer 215 may comprise a separate piece that is secured to a portion of device body 205 and may have a thickness ranging from about 100 microns to about 1.5 mm and is produced from one or more highly thermally conductive materials such as, but not limited to, beryllium oxide, diamond, aluminum nitride, or one or more combinations of these materials.

In another embodiment, second thermally conductive layer 215 may comprise a very thin layer applied over at least a portion of device body 205 (e.g., by chemical or plasma vapor deposition or plasma flame spraying). In such an embodiment, the thickness of second thermally layer 215 may be only about 0.05 micron to about 50 microns. The thickness and surface area of second thermally conductive layer 215 is sufficient to ensure that most, if not essentially all, of the waste heat generated by thermoelectric assembly 212 is transferred through second thermally conductive layer 215 and dissipated into body 205 material. At moderate to low operating temperatures, second thermally conductive layer 215 can dissipate heat from the substrate of thermoelectric assembly at the same rate that heat is dissipated into thermoelectric assembly 212 substrate from thermoelectric assembly 212 thereby allowing continuous moderate to low temperature operation.

In another embodiment, thermoelectric cooling device 227 may contain water or a fan as a heat sink to remove accumulated heat in thermoelectric assembly 212.

In one embodiment, extra-oral thermoelectric assembly 212 comprises different sized thermoelectric plates, types (e.g., high performance, micro, multi-stage, series-parallel or standard) and shapes, and a thermally conductive layer (not shown). In another embodiment, thermally conductive layer may comprise a separate, relatively thick member that is secured to body, rather than being a very thin layer applied by vapor deposition or plasma flame spraying techniques. In a further embodiment, a relatively thin thermally conductive layer is applied to cooling device 227 of the extra-oral thermoelectric anesthetic cooling device (e.g., by vapor deposition or plasma flame spraying).

In one or more embodiments, the exterior surface of cooling device 227 may be coated with one or more coatings to protect the surface and/or facilitate cleaning and/or sterilizing thermoelectric anesthetic cooling device 227. In one or more embodiments, a thermally conductive grease, gel, or adhesive can include a filler material to improve thermal conductivity.

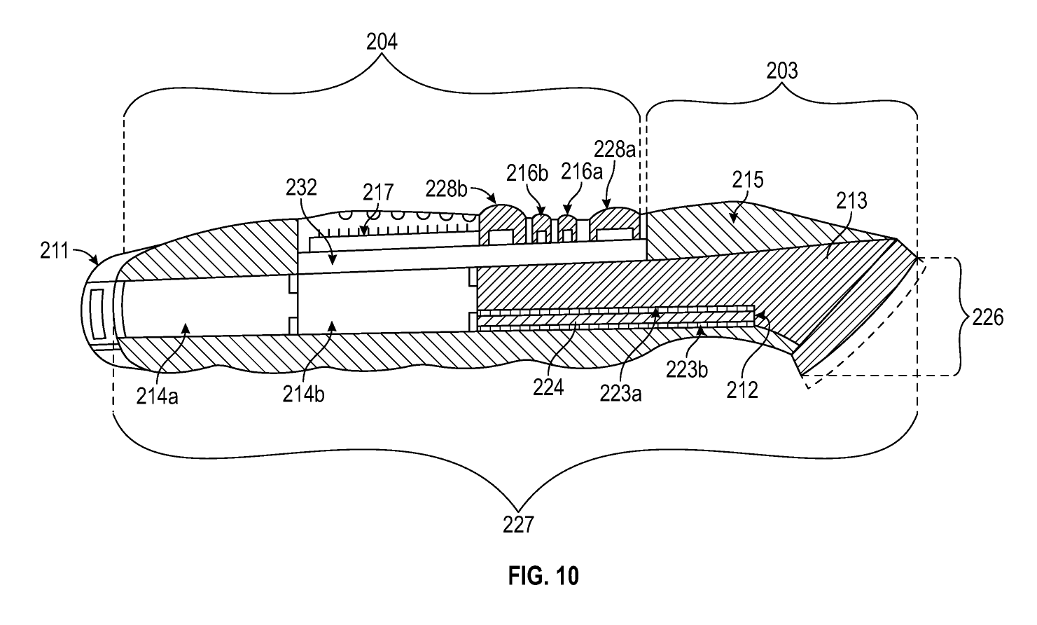

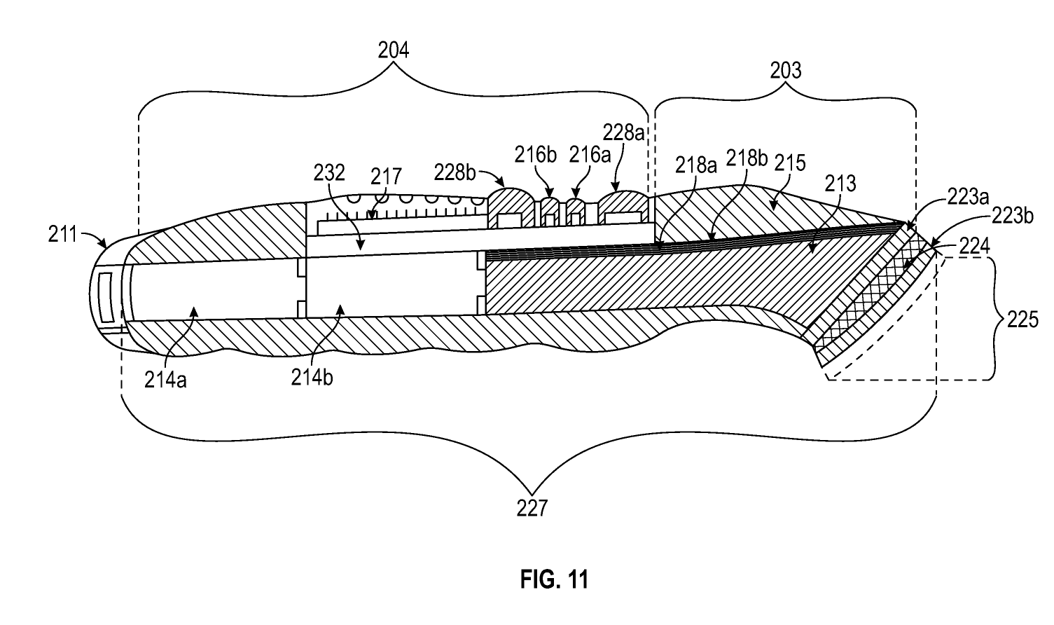

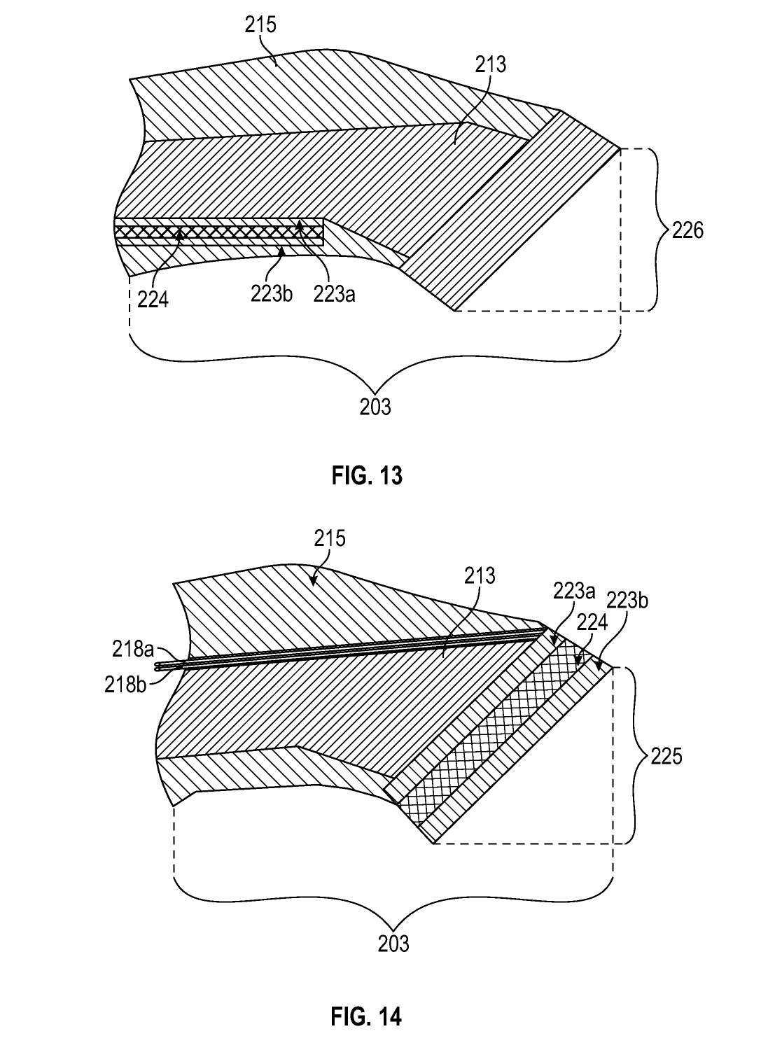

In an exemplary embodiment, as depicted in FIGS. 10 and 13, thermoelectric assembly 212 resides within body 205. In this embodiment, thermoelectric assembly 212 resides in handle 204. In this embodiment, thermoelectric assembly 212 comprises inner ceramic plate 223a and outer ceramic plate 223b, and layer 224 of semiconductor material arranged with spaced apart portions of P-type semiconductor and N-type semiconductor material, wherein layer 224 is disposed between ceramic plates 223a and 223b. In one embodiment, power supplied by rechargeable batteries 214a and 214b, through wires 218a and 218b located in electronic assembly 217, charge carriers and flow to layer 224, giving rise to the Peltier effect, such that cooling is provided at inner ceramic plate 223a and heating is provided at outer ceramic plate 223b. In this embodiment, first thermally conductive layer 213 is located in head section 202 and extends through part of handle 204, as shown in FIG. 11. Furthermore, first thermally conductive layer 213 is adjacent to inner ceramic plate 223a. A user (i.e., clinician) interfaces target tissue on the patient's skin through inner ceramic plate 223a through cold surface 226, as depicted in FIGS. 10 and 13. In an embodiment, thermoelectric assembly 212 comprises a temperature sensor attached to inner ceramic plate 223a electrically coupled to the terminal block (not shown). The heat generated in outer ceramic plate 223b is dissipated from thermoelectric assembly 212 using a second thermally conductive layer 215 located on the exterior of distal head end 203 and the exterior of handle 204. In one embodiment, the peripheral nerve in the skin is cooled from normal body temperature (about 37° C.) down to about 5° C.

In one or more exemplary embodiments, first thermally conductive layer 213 comprises copper, aluminum alloys, and copper heat pipe with or without water. In one or more exemplary embodiments, second outer thermally conductive layer 215 comprises aluminum, copper, aluminum alloys plus an additional layer of conductive material. In these embodiments, the surface area of second thermally conductive layer 215 is sufficiently large that a majority (e.g., substantially all) of the heat conducted away from thermoelectric assembly 212 by second thermally conductive layer 215 is transferred to device body 205.

In another embodiment, thermoelectric cooling device may contain water or a fan as a heat sink to remove accumulated heat in thermoelectric assembly 212.

The present invention also provides a method of selective localized cooling of patient's target tissue by using extra-oral cooling device 227. In an exemplary embodiment, batteries 214a and 214b of extra-oral thermoelectric cooling device 227 are charged by using the plug charger into the electrical outlet or placing thermoelectric cooling device 227 onto a charging dock station which is connected to an outlet. A single use hygienic barrier sleeve is placed onto head section 202 of cooling device 205. In an embodiment, where thermoelectric assembly 212 is within distal head end 203, head section 202 is the placed onto the target tissue (i.e., the extra oral tissue of patient's skin) that needs to be cooled (i.e., anesthetized). Power button 228a is then turned on to start thermoelectric cooling device 227. Optional vibration to provide is used at this point by pressing vibration switch 207. Desired temperature is reached by using 216a (to increase the temperature) or by using 216b (to decrease the temperature). The operation time is set by using time/mode button 228b. Timing indicator light 210 turns green to show desired time interval. Thermoelectric cooling device 205 operates at 45-second bursts and not continuously. Button 228b shows the power level; an orange light in warning indicator lights 209 indicates a low battery level and a red light indicates device 205 needs to be cooled down.

The current provided by the batteries in electronic assembly 217, flows through device 205 and transferred to thermoelectric assembly 212 by wires 218a and 218b, causing outer ceramic thermoelectric plate 223b to cool down to 4° C. (or other desired temperature as indicated on temperature display 208). The clinician places cold surface 225 of outer ceramic plate 223b on the skin surface of the target tissue causing the target tissue to become numb. The aforementioned method is repeated as needed for any site on the body of the patient. Power button 228a is pressed to turn extra oral thermoelectric cooling device 227 off. The hygienic sleeve is then removed and cooling device is wiped down with sanitizing solution before next patient.

Thus, specific embodiments of an intra-oral and extra-oral thermoelectric cooling devises and methods to employ such devices have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A thermoelectric anesthetic cooling device comprising:
i) a device body comprising:
a) a handle;
b) a distal head end, wherein said distal head end comprising a head section and a neck section;
c) a proximal end; and
d) a plurality of thermally conductive layers for dissipating heat from said cooling device;
ii) a thermoelectric assembly, wherein:
said thermoelectric assembly comprises a plurality of thermoelectric plates; and
said thermoelectric assembly is configured to cool a patient skin target tissue; and
iii) an electronic assembly configured to energize said thermoelectric assembly, wherein said electronic assembly is electrically interconnected to said thermoelectric assembly; and
wherein said head section is configured to support or contain said thermoelectric assembly.

2. The thermoelectric anesthetic cooling device of claim 1, wherein said device is an intra-oral device.

3. The thermoelectric anesthetic cooling device of claim 1, wherein said device is an extra-oral device.

4. The thermoelectric anesthetic cooling device of claim 1, wherein said device body comprises a continuous body, wherein:
said handle, said head section, and said distal head end together comprise a single piece of material.

5. The thermoelectric anesthetic cooling device of claim 1, wherein said plurality of thermoelectric plates comprises:
i) at least one inner thermoelectric plate;
ii) at least one outer thermoelectric plate; and
iii) a layer of semiconductor material arranged with spaced apart portions of P-type semiconductor and N-type semiconductor material, wherein said layer is disposed between said at least one inner thermoelectric plate and said at least one outer thermoelectric plate.

6. The thermoelectric anesthetic cooling device of claim 5, wherein said at least one outer thermoelectric plate provides cooling, and wherein a user contacts a patient's target skin tissue with said at least one outer thermoelectric plate.

7. The thermoelectric anesthetic cooling device of claim 1, wherein said plurality of thermoelectric plates comprises ceramic, glass, porcelain, or composite polymer; and said thermoelectric plates are cascaded, are in a multi-stage thermoelectric cooler, or are stacked on top of each other.

8. The thermoelectric anesthetic cooling device of claim 1, wherein said heat generated during operation of said plurality of thermoelectric plates is dissipated by:
a first thermally conductive layer located within interior of said body; and
(ii) a second thermally conductive layer located on the exterior of said distal head end and the exterior of said handle.

9. The thermoelectric anesthetic cooling device of claim 8, wherein said first thermally conductive layer comprises copper, aluminum alloys, water-copper heat pipe, or copper heat pipe; and wherein said second thermally conductive layer comprises tellurium copper rod, aluminum, aluminum alloys, copper, beryllium oxide, diamond, aluminum nitride, or one or more combinations thereof.

10. The thermoelectric anesthetic cooling device of claim 1, wherein said plurality of thermally conductive layers comprises tellurium copper rod, aluminum, magnesium and aluminum alloys, polymers, ceramics, fibers, copper, beryllium oxide, diamond, aluminum nitride, or one or more combinations thereof.

11. The thermoelectric anesthetic cooling device of claim 1, wherein said device further comprises a heat sink selected from a water-copper heat pipe, or a fan for removing accumulated heat in said thermoelectric assembly.

12. A method of selective localized cooling of a patient target tissue using a thermoelectric anesthetic cooling device according to claim 1, said method comprising:
i) energizing said thermoelectric anesthetic cooling device;
ii) activating cooling function of said thermoelectric anesthetic cooling device by energizing said plurality of said thermoelectric plates by said electronic assembly;
iii) placing a cool surface of said thermoelectric plates against said patient target tissue;
iv) cooling said patient target tissue for a desired time and temperature; and
v) removing said device from said patient target tissue.

13. A thermoelectric anesthetic cooling device comprising:
i) a device body comprising:
a) a handle;
b) a distal head end, wherein said distal head end comprises a head section and a neck section;
c) a proximal end; and
d) a plurality of thermally conductive layers for dissipating heat from said cooling device;
ii) a thermoelectric assembly, wherein:
said thermoelectric assembly comprises a plurality of thermoelectric plates; and
said thermoelectric assembly is configured to cool a patient skin target tissue; and
iii) an electronic assembly configured to energize said thermoelectric assembly, wherein said electronic assembly is electrically interconnected to said thermoelectric assembly; and
wherein said handle is configured to support or contain said thermoelectric assembly.

14. The thermoelectric anesthetic cooling device of claim 13, wherein said device is an intra-oral device.

15. The thermoelectric anesthetic cooling device of claim 13, wherein said device is an extra-oral device.

16. The thermoelectric anesthetic cooling device of claim 13, wherein said body comprises a continuous body, wherein:
said handle, said head section and said distal head end comprise a single piece of material.

17. The thermoelectric anesthetic cooling device of claim 13, wherein said plurality of thermoelectric plates comprises:
i) at least one inner thermoelectric plate;
ii) at least one outer thermoelectric plate; and
iii) a layer of semiconductor material arranged with spaced apart portions of P-type semiconductor and N-type semiconductor material, wherein said layer is disposed between said at least one inner thermoelectric plate and said at least one outer thermoelectric plate.

18. The thermoelectric anesthetic cooling device of claim 17, wherein said at least one inner thermoelectric plate provides cooling; said at least one inner thermoelectric plate is adjacent to a first thermally conductive layer; and wherein a user contacts a patient's target skin tissue through a cool surface at said head section of said device.

19. The thermoelectric anesthetic cooling device of claim 13, wherein said plurality of thermoelectric plates comprises ceramic, glass, porcelain or composite polymer, and said thermoelectric plates are cascaded, are in a multi-stage thermoelectric cooler, or are stacked on top of each other.

20. The thermoelectric anesthetic cooling device of claim 13, wherein said heat generated during operation of said plurality of thermoelectric plates is dissipated by a second thermally conductive layer located on the exterior of said distal head end and the exterior of said handle.

21. The thermoelectric anesthetic cooling device of claim 18, wherein said first thermally conductive layer comprises copper, aluminum alloys, water-copper heat pipe, or copper heat pipe.

22. The thermoelectric anesthetic cooling device of claim 20, wherein said second thermally conductive layer comprises tellurium copper rod, aluminum, aluminum alloys, copper, beryllium oxide, diamond, aluminum nitride, or one or more combinations thereof.

23. The thermoelectric anesthetic cooling device of claim 13, wherein said plurality of thermally conductive layers comprises tellurium copper rod, aluminum, magnesium and aluminum alloys, polymers, ceramics, fibers, copper, beryllium oxide, diamond, aluminum nitride, or one or more combinations thereof.

24. The thermoelectric cooling device of claim 13, wherein said device further comprises a heat sink selected from a water-copper heat pipe, and a fan for removing accumulated heat in said thermoelectric assembly.

25. A method of selective localized cooling of patient target tissue by using a thermoelectric anesthetic cooling device according to claim 13, comprising:
i) energizing said cooling device;
ii) activating cooling function of said thermoelectric anesthetic cooling device by energizing said plurality of said thermoelectric plates by said electronic assembly;
iii) placing a cool surface of said thermoelectric plates against said patient target tissue;
iv) cooling said patient target tissue for a desired time and temperature; and
v) removing said thermoelectric anesthetic cooling device from said patient target tissue.

26. An intra-oral thermoelectric anesthetic cooling device comprising:
i) a device body comprising:
a) a handle;
b) a distal head end, wherein said distal head end comprises a head section, and a neck section;
c) a proximal end; and
d) a plurality of thermally conductive layers for dissipating heat from said thermoelectric anesthetic cooling device; and
wherein said body comprises a continuous body, wherein:
said handle, said head section and said distal head end comprise a single piece of material; and
ii) a thermoelectric assembly, wherein:
said thermoelectric cooling assembly comprises a plurality of thermoelectric plates, wherein said plurality of thermoelectric plates comprises:
a) at least one inner thermoelectric plate;
b) at least one outer thermoelectric plate; and
c) a layer of semiconductor material arranged with spaced apart portions of P-type semiconductor and N-type semiconductor material, wherein said layer is disposed between said at least one inner thermoelectric plate and said at least one outer thermoelectric plate;
wherein said at least one outer thermoelectric plate provides cooling, and wherein a user contacts a patient's skin target tissue through said at least one outer thermoelectric plate; and
said thermoelectric assembly is to configured to cool a patient's target skin surface; and
iii) an electronic assembly configured to energize said thermoelectric assembly, wherein said electronic assembly is electrically interconnected to said thermoelectric assembly; and
wherein said head section is configured to support or contain said thermoelectric assembly.

27. The thermoelectric anesthetic cooling device of claim 26, wherein said plurality of thermoelectric plates comprises ceramic, glass, porcelain or composite polymer; and said thermoelectric plates are cascaded, are in a multi-stage thermoelectric cooler, or are stacked on top of each other.

28. The thermoelectric anesthetic cooling device of claim 26, wherein said heat generated during operation of said plurality of thermoelectric plates is dissipated by:
(i) a first thermally conductive layer located within interior of said body; and
(ii) a second thermally conductive layer located on the exterior of said distal head end and the exterior of said handle.

29. The thermoelectric anesthetic cooling device of claim 28, wherein said first thermally conductive layer comprises copper, aluminum alloys, water-copper heat pipe, or copper heat pipe; and wherein said second thermally conductive layer comprises tellurium copper rod, aluminum, aluminum alloys, copper, beryllium oxide, diamond, aluminum nitride, or one or more combinations thereof.

30. The thermoelectric anesthetic cooling device of claim 26, wherein said plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

31. The thermoelectric anesthetic cooling device of claim 26, wherein said device further comprises a heat sink selected from a water-copper heat pipe, and a fan for removing accumulated heat in said thermoelectric assembly.

32. An intra-oral thermoelectric anesthetic cooling device comprising:
i) a device body comprising:
a) a handle;
b) a distal head end, wherein said distal head end comprises a head section and a neck section;
c) a proximal end; and
d) a plurality of thermally conductive layers for dissipating heat from said cooling device; and
wherein said body comprises a continuous body, wherein:
said handle, said head section and said distal head end comprise a single piece of material; and
ii) a thermoelectric assembly, wherein:
said thermoelectric cooling assembly comprises a plurality of thermoelectric plates, wherein said plurality of thermoelectric plates comprises:
a) at least one inner thermoelectric plate;
b) at least one outer thermoelectric plate; and
c) a layer of semiconductor material arranged with spaced apart portions of P-type semiconductor and N-type semiconductor material, wherein said layer is disposed between said at least one inner thermoelectric plate and said at least one outer thermoelectric plate;
wherein said at least one inner thermoelectric plate provides cooling, said at least one inner thermoelectric plate is adjacent to a first thermally conductive layer; and wherein a user contacts said patient's target skin tissue through a cool surface at said head section of said device; and
said thermoelectric assembly is configured to cool a patient's target skin surface; and
iii) an electronic assembly configured to energize said thermoelectric assembly, wherein said electronic assembly is electrically interconnected to said thermoelectric assembly;
wherein said handle is configured to support or contain said thermoelectric assembly.

33. The thermoelectric cooling device of claim 32, wherein said plurality of thermoelectric plates comprises ceramic, glass, porcelain or composite polymer, and said thermoelectric plates are cascaded, are in a multi-stage thermoelectric coolers, or are stacked on top of each other.

34. The thermoelectric cooling device of claim 32, wherein said heat generated during operation of said plurality of thermoelectric plates is dissipated by a second thermally conductive layer located on the exterior of said distal head end and the exterior of said handle.

35. The thermoelectric cooling device of claim 32, wherein said first thermally conductive layer comprises copper, aluminum alloys, water-copper heat pipe, or copper heat pipe.

36. The thermoelectric cooling device of claim 34, wherein said second thermally conductive layer comprises tellurium copper rod, aluminum, aluminum alloys, copper, beryllium oxide, diamond, aluminum nitride, or one or more combinations thereof.

37. The thermoelectric cooling device of claim 32, wherein said plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

38. The thermoelectric cooling device of claim 32, wherein said device further comprises a heat sink selected from a water-copper heat pipe, and a fan for removing accumulated heat in said thermoelectric assembly.

39. An extra-oral thermoelectric anesthetic cooling device comprising:
i) a device body comprising:
a) a handle;
b) a distal head end, wherein said distal head end comprises a head section, and a neck section;
c) a proximal end; and
d) a plurality of thermally conductive layers for dissipating heat from said cooling device; and
wherein said body comprises a continuous body, wherein:
said handle, said head section and said distal head end comprise a single piece of material; and
ii) a thermoelectric assembly, wherein:
said thermoelectric assembly comprises a plurality of thermoelectric plates, wherein said plurality of thermoelectric plates comprises:
a) at least one inner thermoelectric plate;
b) at least one outer thermoelectric plate; and
c) a layer of semiconductor material arranged with spaced apart portions of P-type semiconductor and N-type semiconductor material, wherein said layer is disposed between said at least one inner thermoelectric plate and said at least one outer thermoelectric plate;
wherein said at least one outer thermoelectric plate provides cooling, and wherein a user contacts said patient's target skin tissue through said at least outer thermoelectric plate; and
said thermoelectric assembly is configured to cool a patient's target skin surface; and
iii) an electronic assembly configured to energize said thermoelectric assembly, wherein said electronic assembly is electrically interconnected to said thermoelectric assembly; and
wherein said head section is configured to support or contain said thermoelectric assembly.

40. The thermoelectric anesthetic cooling device of claim 39, wherein said plurality of thermoelectric plates comprises ceramic, glass, porcelain or composite polymer; and
said thermoelectric plates are cascaded, are in a multi-stage thermoelectric cooler, or are stacked on top of each other.

41. The thermoelectric cooling device of claim 39, wherein said heat generated during operation of said plurality of thermoelectric plates is dissipated by:
(i) a first thermally conductive layer located within interior of said body; and
(ii) a second thermally conductive layer located on the exterior of said distal head end and the exterior of said handle.

42. The thermoelectric anesthetic cooling device of claim 41, wherein said first thermally conductive layer comprises copper, aluminum alloys, water-copper heat pipe, or copper heat pipe; and wherein said second thermally conductive layer comprises tellurium copper rod, aluminum, aluminum alloys, copper, beryllium oxide, diamond, aluminum nitride, or one or more combinations thereof.

43. The thermoelectric anesthetic cooling device of claim 39, wherein said plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

44. The thermoelectric anesthetic cooling device of claim 39, wherein said device further comprises a heat sink selected from a water-copper heat pipe, and a fan for removing accumulated heat in said thermoelectric assembly.

45. An extra-oral thermoelectric anesthetic cooling device comprising:
i) a device body comprising:
a) a handle;
b) a distal head end, wherein said distal head end comprises a head section and a neck section;
c) a proximal end; and
d) a plurality of thermally conductive layers for dissipating heat from said cooling device; and
wherein said body comprises a continuous body, wherein:
said handle, said head section and said distal head end comprise a single piece of material; and
ii) a thermoelectric assembly, wherein:
said thermoelectric assembly comprises a plurality of thermoelectric plates, wherein said plurality of thermoelectric plates comprises:
a) at least one inner thermoelectric plate;
b) at least one outer thermoelectric plate; and
c) a layer of semiconductor material arranged with spaced apart portions of P-type semiconductor and N-type semiconductor material, wherein said layer is disposed between said at least one inner thermoelectric plate and said at least one outer thermoelectric plate, wherein said at least one inner thermoelectric plate provides cooling; said at least one inner thermoelectric plate is adjacent to a first thermally conductive layer; and wherein a user contacts said patient's target skin tissue through a cool surface at said head section of said device; and
said thermoelectric assembly is configured to cool a patient's skin target surface; and
iii) an electronic assembly configured to energize said thermoelectric assembly, wherein said electronic assembly is electrically interconnected to said thermoelectric assembly,
wherein said handle is configured to support or contain said thermoelectric assembly.

46. The thermoelectric anesthetic cooling device of claim 45, wherein said plurality of thermoelectric plates comprises ceramic, glass, porcelain or composite polymer, and said thermoelectric plates are cascaded, are in a multi-stage thermoelectric coolers, or are stacked on top of each other.

47. The thermoelectric anesthetic cooling device of claim 45, wherein said heat generated during operation of said plurality of thermoelectric plates is dissipated by a second thermally conductive layer located on the exterior of said distal head end and the exterior of said handle.

48. The thermoelectric anesthetic cooling device of claim 45, wherein said first thermally conductive layer comprises copper, aluminum alloys, water-copper heat pipe, or copper heat pipe.

49. The thermoelectric anesthetic cooling device of claim 47, wherein said second thermally conductive layer comprises tellurium copper rod, aluminum, copper, aluminum alloys, beryllium oxide, diamond, aluminum nitride, or one or more combinations thereof.

50. The thermoelectric anesthetic cooling device of claim 45, wherein said plurality of thermally conductive layers comprises tellurium copper rod, aluminum, magnesium and aluminum alloys, copper, polymers, ceramics, fibers, beryllium oxide, diamond, aluminum nitride, or one or more combinations thereof.

51. The thermoelectric anesthetic cooling device of claim 45, wherein said device further comprises a heat sink selected from a water-copper heat pipe, and a fan for removing accumulated heat in said thermoelectric assembly.
